(19) United States
(12) Patent Application Publication
(10) Pub. No.: US 2004/0192658 A1
(43) Pub. Date: Sep. 30, 2004

(54) Compositions and methods of using collajolie

(75) Inventors: William L. Hunter, Vancouver (CA); David M. Gravett, Vancouver (CA); Philip M. Toleikis, Vancouver (CA); Arpita Maiti, Vancouver (CA)

Correspondence Address: SEED INTELLECTUAL PROPERTY LAW GROUP PLLC, 701 FIFTH AVE, SUITE 6300, SEATTLE, WA, 98104-7092, US

(73) Assignee: Angiotech International AG, Zug (CH)

(21) Appl. No.: 10/746,911

(22) Filed: Dec. 24, 2003

Related U.S. Application Data
(60) Provisional application No. 60/436,806, filed on Dec. 27, 2002.

(51) Int. Cl. A61K 31/19; A61K 31/65

(52) U.S. Cl. 514/152; 424/94.1; 514/575

(57) ABSTRACT

Compositions and devices including collagen and a metalloprotease inhibitor, and methods of making and using same.

### CROSS-REFERENCE TO RELATED APPLICATION

This application claims the benefit of U.S. Provisional Patent Application No. 60/436,806 filed Dec. 27, 2002, where this provisional application is incorporated herein by reference in its entirety.

### BACKGROUND OF THE INVENTION

1. Field of the Invention

The present invention relates generally to pharmaceutical compositions, devices and methods, and more specifically, to compositions, devices and methods related to enhancing the duration and activity of implanted collagen materials.

2. Description of the Related Art

Collagen is one of the most abundant proteins in mammals, representing up to 30% of the dry weight of the human body (see, L. C. Junqueira and J. Cameiro, Basic Histology, 4th ed., Lange Medical Publications, Los Altos, Calif., 1983, pp. 89-119). Collagen provides strength and flexibility for skin, hair and nails, and is also a major and essential component of muscles, tendons, cartilage, ligaments, joints and blood vessels.

Collagen can be found in at least five different naturally occurring forms that are produced by several different cell types. Type I collagen is the most abundant form of collagen, and can be found throughout the body. It is produced by fibroblasts, osteoblasts, odontoblasts, and chondroblasts, and can be found in bones, dentin, dermis, and fibrous cartilage. Type II collagen is produced by chondroblasts, and can be found primarily in cartilage. Type III collagen is produced by smooth muscle fibroblasts, reticular cells, Schwann cells, and hepatocytes. Its primary function is to maintain the structure of organs, and can be found in smooth muscles, endoneurium, arteries, uterus, liver, spleen, kidney, and lung tissue. Type IV collagen is primarily believed to be involved in support and filtration, and can be found in the epithelial and endothelial basal lamina and basement membranes. Type V collagen is found in fetal membranes, blood vessels, and placental basement membrane.

Collagen has been suggested for use in the treatment of a variety of medical applications, including for example, cosmetic surgery, arthritis, skin regeneration, implants, organ replacement, and treatment for wounds and burns (see e.g., U.S. Pat. Nos. 6,309,670, 5,925,736, 5,856,446, 5,843,445, 5,800,811, 5,783,188, 5,720,955, 5,383,930, 5,106,949, 5,104,660, 5,081,106, 4,837,379, 4,604,346, 4,485,097, 4,546,500, 4,539,716, and 4,409,332) and provides an attractive alternative to the use of injectable botulinum toxin drugs, such as BOTOX® (Allergan, Inc., Irvine, Calif.).

Collagen however, has presented several problems associated with medical applications. For example, in the context of implants, collagen preparations with impurities are potent immunogens that can stimulate an inflammatory response. Similarly, non-human forms of collagen such as bovine collagen have been associated with a chronic cellular inflammatory reaction that can result in scar tissue and adhesion formation, and transient low-grade fevers. In addition, the duration of implantable collagen is limited, requiring procedures (especially for cosmetic enhancement) to be repeated on a regular basis.

The present invention addresses shortcomings associated with collagen and the use thereof in medical applications.

### BRIEF SUMMARY OF THE INVENTION

Briefly stated, the present invention provides compositions, devices, and methods for prolonging the activity of collagen-based implants. Collagen-based implants are used to provide structure and support in a variety of medical procedures including dermal injections for cosmetic purposes (to reduce wrinkles, scars, contour defects), periurethral bulking agents for the management of incontinence, and vascular “plugs” to produce hemostasis following vascular puncture procedures. While extremely effective, collagen implants have a short duration of activity in vivo because the material is rapidly broken down by degradative enzymes (principally collagenase and other matrix metalloproteinase enzymes) released by white blood cells and connective tissue cells (fibroblasts) adjacent to the implant. The result is that the collagen implant procedure must be repeated at frequent intervals to maintain the desired affect.

The present invention describes compositions that combine collagen and a compound that inhibits the activity of collagenase to produce a collagen-based implant with enhanced durability in vivo (“Collajolie”). A variety of naturally occurring and synthetically created molecules are known to inhibit collagenase activity, and have been used for purposes other than that of the present invention (e.g., the treatment of malignancy, arthritis and other disorders) where these inhibitors are collectively known as “matrix metalloproteinase inhibitors” (abbreviated as MMP inhibitors, or MMPIs) or “collagenase inhibitors”. The metalloprotease enzymes have been divided into recognized classes based on common features, where examples are: MMP-1 (collagenase I, fibroblast collagenase; EC 3.4.24.3); MMP-2 (gelatinase A, 72 kDa gelatinase, basement membrane collagenase, EC 3.4.24.24), MMP-3 (stromelysin 1, EC 3.4.24.17); MMP-7 (proteoglycanase, matrilysin); MMP-8 (collagenase II, neutrophil collagenase, EC 3.4.24.34); MMP-9 (gelatinase B, 92 kDa gelatinase, EC 3.4.24.35), MMP-10 (stromelysin 2, EC 3.4.24.22), MMP-11 (stromelysin 3), MMP-12 (metalloelastase, HME, human macrophage elastase); MMP-13 (collagenase III); and MMP-14 (membrane MMP). The present invention is directed to inhibiting MMPs that degrade collagen. Representative examples of MMPI suitable for use within the present invention include TIMP-1, tetracycline, doxycycline, minocycline, BATIMASTAT, MARIMASTAT, RO-1130830, CGS 27023A, BMS-275291, CMT-3, SOLIMASTAT, ILOMASTAT, CP-544439, PRINOMASTAT, PNU-1427690, SU-5402 and TROCADE. Within further embodiments, the compositions described herein may also further comprise one or more factors, compounds or agents which encourage or enhance bone growth, including for example, hydroxyapatite and bone morphogenic proteins (BMP, e.g., BMP-2).

Hence, within one aspect of the present invention compositions are provided comprising collagen and a matrix metalloprotease inhibitor (MMPI). In some aspects, the compositions may further include hydroxyapatite. Within certain embodiments the MMPI is a Tissue Inhibitor of Matrix Metalloproteinase (e.g., TIMP-1, TIMP-2, TIMP-3, or, TIMP-4). Within other embodiments, the MMPI is tetracycline, or an analog or derivative thereof (e.g., minocycline or doxycline); a hydroxamate (e.g., BATIMISTAT, MARIMISTAT, or TROCADE); RO-1130830, CGS 27023A, BMS-275291, CMT-3, SOLIMASTAT, ILOMASTAT, CP-544439, PRINOMASTAT, PNU-1427690, or SU-5402. In other aspects, the MMPI may be a polypeptide inhibitor (e.g., an inhibitor of a metalloprotease maturase), a mercapto-based compound, or a bisphosphonate with structure (I):

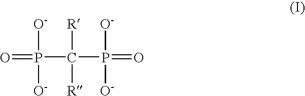

wherein R′ and R″ are independently a hydrogen, a halogen, a hydroxy, an amino group or a substituted derivative thereof, a thio group or a substituted derivative thereof, or an alkyl, alkanyl, alkenyl, alkynyl, alkyldiyl, alkyleno, heteroalkyl, heteroalkanyl, heteroalkenyl, heteroalkanyl, heteroalkyldiyl, heteroalkyleno, aryl, arylalkyl, heteroaryl, or heteroarylalkyl group, or a substituted derivative thereof. In one embodiment, R′ and R″ are independently hydroxy, hydrogen, or chlorine.

Within one aspect, the invention provides a composition that includes collagen, hydroxyapatite, and at least two MMPIs. For example, the composition may include a tetracycline, or an analog or derivative thereof and a bisphosphonate. In another embodiment, the composition includes a tetracycline, or an analog or derivative thereof and a hydroxymate.

Within another aspect, the instant compositions may include collagen, at least one MMPI, and at least one polymer. In one aspect, the polymer is a biodegradable polymer (e.g., albumin, gelatin, starch, cellulose, dextrans, polysaccharides, fibrinogen, poly (esters), poly (D,L lactide), poly (D,L-lactide-co-glycolide), poly (glycolide), poly(ε-caprolactone), poly (hydroxybutyrate), poly (alkylcarbonate), poly(anhydrides), and poly (orthoesters), and copolymers and blends thereof), while in another aspect the polymer is a non-biodegradable polymer (e.g., an ethylene oxide and propylene oxide copolymer, an ethylene vinyl acetate copolymer, silicone rubber, a poly (methacrylate) based polymer, or a poly (acrylate) based polymer).

Within separate embodiments the collagen is a type I collagen or is a type II collagen. Within yet other separate embodiments the compositions provided herein may contain other compounds or compositions, including for example, thrombin and/or dyes, or a bone morphogenic protein, such as BMP-2 or BMP-8. Within further embodiments, the composition may be sterile, and the compositions may be sterilized in a manner suitable for human administration.

The compositions described herein may be utilized for a variety of indications, including for example, as a medical device to augment bone growth, in spinal fusion surgery, as a surgical sling, mesh, or patch, for the treatment of periodontal disease (e.g., as a dental implant), as a skin graft (e.g., for the development of artificial skin), as a corneal shield, or as a glaucoma drainage device. For example, the medical device may be a collagen sponge that includes an MMPI (for representative discussions of collagen sponges, see, e.g., U.S. Pat. Nos. 6,649,162; 6,425,918; 6,183,496; 5,116,552; 4,789,401; 4,412,947; and 4,193,813, as well as Burton et al., British J. of Dermatology 99:681-5, 1978 and Natsume, et al. J. Biomedical Materials Res., 27:867-875, 1993). In certain embodiments, the device may further include a polymer, as described above.

Within other aspects of the present invention, methods are provided for making the compositions described herein, comprising the step of mixing a collagen and one or more MMPI as described herein, preferably in combination with one or more factors, agents or compounds which encourage or assist bone growth including, for example, hydroxyapatite and bone morphogenic proteins (BMP, e.g., BMP-2 and BMP-8). Within further embodiments, the compositions and devices provided herein may be sterilized.

Methods for treating or preventing a variety of indications are provided herein. In one aspect, a method for surgically fusing a portion of a spine is described including removing a portion of a degenerated disc from the spine of a patient to form a disc space, and inserting into the disc space a medical device (either with or without hydroxyapatite) such as described herein. The device may include, e.g., 0.001% to 15% of the MMPI by weight. In another aspect, a method for augmenting bone or replacing lost bone is described. The method includes delivering to a patient in need thereof at a desired location a composition including collagen, an MMPI, and hydroxyapatite. In another aspect, the invention provides a method of treating periodontal disease comprising placing a dental implant that includes collagen and an MMPI (either with or without hydroxyapatite) between gingival tissue and a debrided periodontal defect in the mouth of a patient in need thereof.

Yet other indications may be treated with the use of compositions including collagen and an MMPI according to the present invention. For example, a method of treating gastroesophageal reflux disease is described that includes injecting the composition in accordance with the invention into the vicinity of the lower esophageal sphincter of a patient. In yet another aspect, a method of treating fecal incontinence is described that includes injecting the composition into the vicinity of the anal sphincter of a patient. In yet another aspect, the instant invention provides a method of reinforcing soft tissue during an operative repair (e.g., an abdominal or thoracic wall repair, a hernia repair, a suture line reinforcement, an ostomy reinforcement, a tissue flap donor site repair, or a repair of a tendon, ligament, or cartilage) comprising attaching to the soft tissue a surgical patch that includes collagen and an MMPI (see, e.g., U.S. Pat. Nos. 6,238,416; 5,665,114; and 5,290,217 for representative discussions of surgical patches). In a further aspect, the invention provides a method of improving drainage of the aqueous humor following a sclerectomy comprising inserting into a subscleral drainage channel a glaucoma drainage device that includes collagen and an MMPI. In yet another aspect, a method of improving wound healing is provided that includes applying a wound dressing that includes collagen and an MMPI to a wound surface. In yet another aspect, the present invention describes a method of improving post-operative healing of cornea following cataract surgery comprising applying a corneal shield that includes collagen and an MMPI to scleral or conjunctival tissue.

These and other aspects of the present invention will become evident upon reference to the following detailed description.

### DETAILED DESCRIPTION OF THE INVENTION

Prior to setting forth the invention, it may be helpful to an understanding thereof to set forth definitions of certain terms that will be used hereinafter. “Collagen” as used herein refers to all forms of collagen as are described or referenced herein, including those that have been processed or modified. Representative examples include type I and type II collagen. Collagen may be prepared from human or animal sources, or, may be produced using recombinant techniques. “Matrix Metalloproteinase Inhibitor” or “MMPI” refers to a compound, agent or composition that inhibits matrix metalloproteinase activity. Representative examples of MMP Inhibitors include Tissue Inhibitors of Metalloproteinases (TIMPs) (e.g., TIMP-1, TIMP-2, TIMP-3, or TIMP-4), 0₂-macroglobulin, tetracyclines (e.g., tetracycline, minocycline, and doxycycline), hydroxamates (e.g., BATIMASTAT, MARIMISTAT and TROCADE), chelators (e.g., EDTA, cysteine, acetylcysteine, D-penicillamine, and gold salts), synthetic MMP fragments, succinyl mercaptopurines, phosphonamidates, and hydroxaminic acids.

Any concentration or percentage ranges recited herein are to be understood to include concentrations of any integer within the range and fractions thereof, such as one tenth and one hundredth of an integer, unless otherwise indicated. As used herein, “about” or “comprising essentially of” means ±15%.

Various references are set forth herein which, for example, describe in more detail certain procedures or compositions (e.g., compounds, proteins, vectors, and their generation, etc.). These references, including patents and articles, are incorporated by reference in their entirety. It should also be noted that when a PCT application is referred to it is also understood that the underlying or cited U.S. applications are also incorporated by reference herein in their entirety.

I. Collagen

Collagen is the major component in skin, cartilage, bone, and connective tissue, and occurs in several different types or forms, with Types I, II, III, and IV being most common. Collagen typically is isolated from natural sources, such as bovine bone, cartilage, or hide. Bones are usually defatted, crushed, dried, and demineralized to extract the collagen. In contrast, bovine cartilage or hide is usually minced and digested with enzymes other than collagenase (in order to remove contaminating protein). Collagen can also be prepared from human tissue (the patient's own or donor tissue) or by recombinant methods.

Within certain embodiments of the invention, preferred collagens are prepared as non-immunoreactive sterile compositions. They may be soluble (e.g., VITROGEN collagen in solution, available from Cohesion Technologies, Palo Alto, Calif.), or be in the form of reconstituted fibrillar atelopeptide collagen, for example ZYDERM Collagen Implant available from Inamed Aesthetics, Santa Barbara, Calif.). Other examples of collagens include tissue engineered human collagen products designed for wrinkle reduction, such as COSMODERM, which is intended to treat surface areas, and COSMOPLAST (both from Inamed Aesthetics), which is for treating larger voids, and viscoelastic injectable gel formulations, such as HYLAFORM (Inamed Aesthetics), for the same-day treatment of facial wrinkles and scars.

Representative examples of patents which disclose collagen-containing compositions, devices, and methods for making and/or delivering such compositions and devices include U.S. Pat. Nos. 4,164,559, 4,424,208, 4,140,537, 4,563,350, 4,582,640, 4,642,117, 4,743,229, 4,776,890, 4,795,467, 4,888,366, 5,035,715, 5,162,430, 5,304,595, 5,324,775, 5,328,955, 5,413,791, 5,428,022, 5,446,091, 5,475,052, 5,523,348, 5,527,856, 5,543,441, 5,550,187, 5,565,519, 5,580,923, 5,614,587, 5,616,689, 5,643,464, 5,693,341, 5,744,545, 5,752,974, 5,756,678, 5,786,421, 5,800,541, 5,807,581, 5,823,671, 5,874,500, 5,895,833, 5,936,035, 5,962,648, 6,090,996, 6,096,039, 6,111,165, 6,165,489, 6,166,130, 6,280,727, 6,312,725, and 6,323,278.

II. Matrix Metalloproteinase (MMP) Inhibitors

Metalloproteinases (MMPs) are a group of naturally occurring zinc-dependent enzymes involved in the breakdown and turnover of extracellular matrix macromolecules. Over 23 metalloproteinases have been identified to date and have been broadly categorized into families of enzymes known as collagenases, stromelysins, gelatinases, elastases and matrilysins. Metalloproteinases are derived from a variety of cell types including neutrophils, monocytes, macrophages and fibroblasts.

MMPs are the principle enzymes involved in the breakdown and normal turnover of collagen in vivo. Although numerous MMPs are capable of breaking down several connective tissue elements including collagen, the enzymes with the highest specificity for collagen come from the collagenase family (e.g., MMP-1, MMP-8, MMP-13 and MMP-14). Metalloproteinase activity is inhibited naturally in vivo by a family of inhibitors known as “Tissue Inhibitors of Metalloproteinase” or “TIMPs” which bind to the active region of the metalloproteinase enzyme rendering it inactive. It is the natural balance between enzyme activity and inhibition that regulates the rate of metabolism of the extracellular matrix under physiologic conditions.

Assays for measuring MMP inhibition are readily known in the art, and include, for example, the following: Cawston T. E., Barrett A. J., “A rapid and reproducible assay for collagenase using [¹⁴C] acetylated collagen,” Anal. Biochem. 35:1961-1965 (1963); Cawston T. E., Murphy G. “Mammalian collagenases,” Methods in Enzymology 80:711 (1981); Koshy P. T. J., Rowan A. D., Life P. F., Cawston T. E., “96-well plate assays for measuring collagenase activity using (3)H-acetylated collagen,” Anal. Biochem. 99:340-345 (1979); Stack M. S., Gray R. D., “Comparison of vertebrate collagenase and gelatinase using a new fluorogenic substrate peptide,” J. Biol. Chem. 264:4277-4281 (1989); and Knight C. G, Willenbrock F., Murphy G, “A novel coumarin-labelled peptide for sensitive continuous assays of the matrix metalloproteinases,” FEBS Lett 296:263-266 (1992). These and other assays known in the art are suitably used to identify an MMPI that may be used in the present invention.

Within the context of this invention, an MMPI may have an Inhibitory Concentration (IC) ranging from about 3-10 mM to about 9-10 nM, with preferred concentrations of about 50 mM to about 50 nM.

When collagen is implanted as part of a therapeutic procedure, it is gradually metabolized by enzymes from the MMP family until it is fully resorbed. This gradual loss of structural integrity due to enzymatic degradation of the collagen implant results in loss of functional activity leading to implant failure and, ultimately, the need for subsequent reintervention. Attempts at prolonging the activity of the collagen implant have centered on crosslinking the collagen implant so as to slow enzymatic degradation. The present invention describes incorporating into the collagen implant an agent or agents capable of inhibiting MMP activity so as to tip the physiologic balance in favor of collagen preservation. This invention is compatible with, and can be used in combination with other preservation strategies, such as collagen crosslinking, designed to increase the residence time of a collagen implant.

Since pathologic production of MMPs has been associated with a variety of clinically important disease processes such as tumor metastasis and the progression of chronic inflammatory conditions such as osteoarthritis and rheumatoid arthritis, numerous naturally occurring and synthetic agents have been developed to inhibit MMP activity. Not surprisingly, regulation of MMP activity is an important and highly regulated process in vivo. As a result there are numerous sites in the pathway leading to MMP production where it is possible to develop molecules capable of inhibiting MMP synthesis or activity. The types of agents capable of inhibiting MMP activity are described in more detail below, and may be used according to the compositions, methods and devices of the present invention.

Briefly, a variety of cytokines (e.g., TNF-α, IL-1, FGF and others) are capable of stimulating the pathway which leads to the production of MMPs. Inhibitors of these cytokines or agents which block their cellular receptors have been demonstrated to inhibit MMP synthesis under certain circumstances and are suitable for use in this invention. After binding to its cellular receptor, the stimulus for MMP production triggers the production of a variety of second messengers and cell signaling molecules (e.g., jun kinase, JKK), inhibition of which can also reduce the production of MMPs. A variety of transcription factors (e.g., c-fos, c-jun, NFκ-B, c-myc) have been implicated in the transcription of the MMP genes. Inhibitors of these transcription factors and their products (e.g., the AP-1 protein) can also decrease the amount of MMPs transcribed and can be utilized for the purposes of this invention. Similarly, strategies that inhibit the MMP gene itself (e.g., gene knockout) or MMP RNA (e.g., antisense, ribozymes, tetracycline, doxycycline, minocycline) can be utilized in this invention to decrease the amount of active MMP enzyme in the region surrounding the collagen implant.

In addition, it is possible to inhibit the function and activity of metalloproteinases after they have been secreted from the cell. Since MMPs are secreted from the cell as inactive precursor proteins (called Pro-MMPs) that are subsequently converted to the active enzyme through a highly specific enzymatic cleavage (catalyzed by enzymes such as plasmin, mast cell protease, cathepsin Q plasma kallikrein and others), it is possible to inhibit the conversion of the MMP from its inactive to active state (thereby maintaining it in an inactive form). Inhibitors of the enzymes responsible for the conversion of the MMP from its inactive to active state can also be utilized for this invention. In addition, it is possible to directly inhibit the function of an activated MMP through several mechanisms such as chelation of its zinc metal active center (e.g., EDTA, cysteine, acetylcysteine, D-penicillamine, gold salts; hydroxamates such as BATIMASTAT, MARIMASTAT, TROCADE (F. Hoffman-La Roche Ltd., Basel, Switzerland), Actinonin, Matylystatins; phosphonic acid inhibitors; phosphonates; phosphonamidates; thiols and sulfodiimines which form monodentate coordination with the catalytic zinc; carboxylates which form bidentate coordination with the catalytic zinc; succinyl mercaptoketones and mercaptoalcohols). These compounds are quite effective at inhibiting MMP activity and may be used for the purposes of this invention.

An important class of MMPIs exert their effect through specific binding to the MMP leading to the formation of an inactive complex. These compounds, known as Tissue Inhibitors of Metalloproteinases (TIMPs) such as TIMP-1, TIMP-2, TIMP-3, and TIMP-4, are capable of inhibiting the activity of virtually all of the MMPs. Although any of the TIMPs are suitable for the purposes of this invention, TIMP-1 (and to a lesser extent TIMP-2) is particularly preferred as it has the highest specificity for inhibition of collagenase. It should also be noted that any compound which increases the production of TIMPs may be capable of preserving collagen and, therefore, may be useful in the practice of this invention. Still other inhibitors act by preventing binding of the MMP to its substrate (e.g., synthetic MMP fragments, synthetic collagen fragments) and may be utilized alone, or in combination with other MMPIs for the purpose of this invention. It should be clear to one of skill in the art that regardless of the specific mechanism of inhibition, any agent capable of inhibiting the production, activation or enzymatic function of the MMP enzymes are ideal agents for the purposes of this invention.

Representative examples of MMPIs include actinonin (3-[[1-[[2-(hydroxymethyl)-1-pyrolidinyl]carbamoyl]-octano-hydroxamic acid); bromocyclic-adenosine monophosphate; N-chlorotaurine; BATIMISTAT, also known as BB-94 (British Biotech, UK); CT1166, also known as N1 {N-[2-(morpholinosulphonylamino)-ethyl]-3-cyclohexyl-2-(S)-propanamidyl}-N4-hydroxy-2-(R)-[3-(4-methylphenyl)propyl]-succinamide (Biochem. J. 308:167-175 (1995)); estramustine (estradiol-3-bis(2-chloroethyl)carbamate); eicosa-pentaenoic acid; MARIMASTAT (also known as BB-2516); matlystatin-B; peptidyl hydroxamic acids such as pNH₂-Bz-Gly-Pro-D-Leu-D-Ala-NHOH (Biophys. Biochem. Res. Comm. 199:1442-1446 (1994)); N-phosphonalkyl dipeptides such as N-[N-((R)-1-phosphonopropyl)-(S)-leucyl]-(S)-phenylalanine-N-methylamide (J. Med. Chem. 37:158-169 (1994)); protocatechuic aldehyde (3,4-dihydroxybenzaldehyde); Ro-31-7467, also known as 2-[(5-bromo-2,3-dihydro-6-hydroxy-1,3-dioxo-1H-benz[de]isoquinolin-2-yl)methyl](hydroxy)-[phosphinyl]-N-(2-oxo-3-azacyclotridecanyl)-4-methylvaleramide; tetracyclines such as (4-(dimethylamino)-1,4,4a,5,5a,6,11,12a-octahydro-3,6,10,12,12a-pentahydroxy-6-methyl-1,11-dioxo-2-naphthacenecarboxamide), doxycycline (α-6-deoxy-5-hydroxy-tetracycline) minocycline (7-dimethylamino-6-dimethyl-6-deoxytetracycline), and methacycline (6-methylene oxytetracycline); trifluoroacetate (J. Med Chem. 36:4030-4039 (1993)); and 1,10-phenanthroline (o-phenanthroline [4-(N-hydroxyamino)-2R-isobutyl-3S-(thiopen-2-ylthiomethyl)-succinyl]-L-phenylalanine-N-methylamidecarboxyalkylamino-based compounds such as N-[1-(R)-carboxy-3-(1,3-dihydro-2H-benz[f]isoindol-2-yl)propyl]-N′,N′-dimethyl-L-leucinamide.

Other representative MMPIs include, for example, chelators (e.g., EDTA, cysteine, acetylcysteine, D-penicillamine, and gold salts); bis(dioxopiperzaine), see U.S. Pat. No. 5,866,570; NEOVASTAT (Les Laboratoires Aetema, Inc., Canada), which inhibits gelatinolytic and elastinolytic activities for MMP-2, MMP-9, and MMP-12 (see, e.g., U.S. Pat. No. 6,168,807, Aeterna Laboratorie); KB-R7785 (Akzo Nobel); ILOMASTAT available from Glycomed/Ligand, Inc., (see, e.g., U.S. Pat. No. 5,892,112); RPR-122818 (Aventis S. A., France); SOLIMASTAT (British Biotech; see, e.g., WO 99/25693); BB-1101, BB-2983, BB-3644 (British Biotech); BMS-275291 (see Rizvi et al., Proceedings of the 1999 AACR NCI EORTC International Conference #726 “A Phase I, safety and pharmacokinetic trial of BMS-275291, a matrix metalloproteinase inhibitor (MMPI), in patients with advanced or metastatic cancer”); D-1927, D-5410 Bristol Meyers-Squibb; CH-5902, CH-138 (Celltech Group, UK); CMT-3 (chemically modified tetracycline 3), DERMOSTAT (CollaGenex Pharmaceuticals, Inc., Newtown, Pa.; U.S. Pat. No. 5,837,696); DAC-MMPI (ConjuChem Inc., Canada); RS-1130830 and RS-113-080 (F. Hoffmann-La Roche Ltd., Switzerland); GM-1339 (Ligand Pharmaceuticals, Inc., San Diego, Calif.); GI-155704A (GlaxoSmithKline, UK); ONO-4817 (Ono Pharmaceutical Co., Osaka, Japan); AG-3433, AG-3088, PRINOMASTAT (Agouron Pharmaceuticals, San Diego, Calif.; see, e.g., U.S. Pat. No. 5,753,653), CP-544439 (Pfizer Inc., New York, N.Y.; U.S. Pat. No. 6,156,798); POL-641 (Polifarma SpA, Italy); SC-964, SD-2590, PNU-142769 (Pharmacia Corporation, Peapack, N.J.; WO 97/32846), SU-5402 (Pharmacia; WO 98/50356); PGE-2946979, PGE-4304887 (Procter & Gamble, Cincinnati, Ohio); fibrolase-conjugate (Schering-AG, Berlin, Germany); EF-13 (Scotia-Pharmaceuticals, Scotland); S-3304 (Shionogi, Japan); CGS-25015 and CGS-27023A (Novartis, Switzerland); XR-168 (Xenova, UK); and RO 1130830 (Fisher, et al., 219 American Chemical Society National Meeting, San Francisco, Calif., Mar. 26-30, 2000, ORGN 830 “Synthesis of RO 1130830, a Matrix Metalloproteinase Inhibitor: Evolution of a Research Scheme to Pilot-Plant Production”). Other MMPIs are described, e.g., in U.S. Pat. Nos. 4,235,885; 4,263,293; 4,276,284; 4,297,275; 4,367,233; 4,371,465; 4,371,466; 4,374,765; 4,382,081; 4,558,034; 4,704,383; 4,950,755; 5,270,447, 6,294,694, and 6,329,550.

Additional MMPIs are described as follows, D-9120, BB-2827, BB-1101 (2S-allyl-N1-hydroxy-3R-isobutyl-N4-(1S-methylcarbamoyl-2-phenylethyl)-succinamide), BB-2983, solimastat (N′-[2,2-Dimethyl-1(S)-[N-(2-pyridyl)carbamoyl]propyl]-N4-hydroxy-2(R)-isobutyl-3(S)-methoxysuccinamide), N4-hydroxy-N1-[2-(methylamino)-2-oxo-1-(phenylmethyl)ethyl]-2-(2-methylpropyl)-3-[(2-thienylthio)methyl]-, [2R-[1 (S*),2R*,3 S*]]-[CAS]), rebimastat (L-Valinamide, N-((2S)-2-mercapto-1-oxo-4-(3,4,4-trimethyl-2,5-dioxo-1-imidazolidinyl)butyl)-L-leucyl-N,3-dimethyl-[CAS]), PS-508, CH-715, nimesulide (Methanesulfonamide, N-(4-nitro-2-phenoxyphenyl)-[CAS]), hexahydro-2-[2(R)-[1(RS)-(hydroxycarbamoyl)-4-phenylbutyl]nonanoyl]-N-(2,2,6,6-etramethyl-4-piperidinyl)-3(S)-pyridazine carboxamide, Cipemastat (1-Piperidinebutanamide, β-(cyclopentylmethyl)-N-hydroxy-Gamma-oxo-Alpha-[(3,4,4-trimethyl-2,5-dioxo-1-imidazolidinyl)methyl]-,(AlphaR,βR)-[CAS]), 5-(4′-biphenyl)-5-[N-(4-nitrophenyl)piperazinyl]barbituric acid, 6-methoxy-1,2,3,4-tetrahydro-norharman-1-carboxylic acid, Ro-31-4724 (L-Alanine, N-[2-[2-(hydroxyamino)-2-oxoethyl]-4-methyl-1-oxopentyl]-L-leucyl-, ethyl ester[CAS]), N-hydroxy-2,2-dimethyl-4-((4-(4-pyridinyloxy)phenyl)sulfonyl)-, (3R)-[CAS]), PNU-142769 (2H-Isoindole-2-butanamide, 1,3-dihydro-N-hydroxy-Alpha-[(3S)-3-(2-methylpropyl)-2-oxo-1-(2-phenylethyl)-3-pyrrolidinyl]-1,3-dioxo-, (AlphaR)-[CAS]), (S)-1-[2-[[[(4,5-Dihydro-5-thioxo-1,3,4-thiadiazol-2-yl)amino]-carbonyl]amino]-1-oxo-3-(pentafluorophenyl)propyl]-4-(2-pyridinyl)piperazine, SC-77964, PNU-171829, N-hydroxy-2(R)-[(4-methoxybenzene-sulfonyl)(4-picolyl)amino]-2-(2-tetrahydrofuranyl)-acetamide, L-758354 ((1,1′-Biphenyl)-4-hexanoic acid, Alpha-butyl-Gamma-(((2,2-dimethyl-1-((methylamino)carbonyl)propyl)amino)carbonyl)-4′-fluoro-, (AlphaS-(AlphaR*,GammaS*(R*)))-[CAS]), or an analogue or derivative thereof.

Additional representative examples of MMPIs are identified in U.S. Pat. Nos. 5,665,777; 5,985,911; 6,288,261; 5,952,320; 6,441,189; 6,235,786; 6,294,573; 6,294,539; 6,563,002; 6,071,903; 6,358,980; 5,852,213; 6,124,502; 6,160,132; 6,197,791; 6,172,057; 6,288,086; 6,342,508; 6,228,869; 5,977,408; 5,929,097; 6,498,167; 6,534,491; 6,548,524; 5,962,481; 6,197,795; 6,162,814; 6,441,023; 6,444,704; 6,462,073; 6,162,821; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 5,861,436; 5,691,382; 5,763,621; 5,866,717; 5,902,791; 5,962,529; 6,017,889; 6,022,873; 6,022,898; 6,103,739; 6,127,427; 6,258,851; 6,310,084; 6,358,987; 5,872,152; 5,917,090; 6,124,329; 6,329,373; 6,344,457; 5,698,706; 5,872,146; 5,853,623; 6,624,144; 6,462,042; 5,981,491; 5,955,435; 6,090,840; 6,114,372; 6,566,384; 5,994,293; 6,063,786; 6,469,020; 6,118,001; 6,187,924; 6,310,088; 5,994,312; 6,180,611; 6,110,896; 6,380,253; 5,455,262; 5,470,834; 6,147,114; 6,333,324; 6,489,324; 6,362,183; 6,372,758; 6,448,250; 6,492,367; 6,380,258; 6,583,299; 5,239,078; 5,892,112; 5,773,438; 5,696,147; 6,066,662; 6,600,057; 5,990,158; 5,731,293; 6,277,876; 6,521,606; 6,168,807; 6,506,414; 6,620,813; 5,684,152; 6,451,791; 6,476,027; 6,013,649; 6,503,892; 6,420,427; 6,300,514; 6,403,644; 6,177,466; 6,569,899; 5,594,006; 6,417,229; 5,861,510; 6,156,798; 6,387,931; 6,350,907; 6,090,852; 6,458,822; 6,509,337; 6,147,061; 6,114,568; 6,118,016; 5,804,593; 5,847,153; 5,859,061; 6,194,451; 6,482,827; 6,638,952; 5,677,282; 6,365,630; 6,130,254; 6,455,569; 6,057,369; 6,576,628; 6,110,924; 6,472,396; 6,548,667; 5,618,844; 6,495,578; 6,627,411; 5,514,716; 5,256,657; 5,773,428; 6,037,472; 6,579,890; 5,932,595; 6,013,792; 6,420,415; 5,532,265; 5,691,381; 5,639,746; 5,672,598; 5,830,915; 6,630,516; 5,324,634; 6,277,061; 6,140,099; 6,455,570; 5,595,885; 6,093,398; 6,379,667; 5,641,636; 5,698,404; 6,448,058; 6,008,220; 6,265,432; 6,169,103; 6,133,304; 6,541,521; 6,624,196; 6,307,089; 6,239,288; 5,756,545; 6,020,366; 6,117,869; 6,294,674; 6,037,361; 6,399,612; 6,495,568; 6,624,177; 5,948,780; 6,620,835; 6,284,513; 5,977,141; 6,153,612; 6,297,247; 6,559,142; 6,555,535; 6,350,885; 5,627,206; 5,665,764; 5,958,972; 6,420,408; 6,492,422; 6,340,709; 6,022,948; 6,274,703; 6,294,694; 6,531,499; 6,465,508; 6,437,177; 6,376,665; 5,268,384; 5,183,900; 5,189,178; 6,511,993; 6,617,354; 6,331,563; 5,962,466; 5,861,427; 5,830,869; 6,087,359.

Representative examples of classes of MMPIs which are discussed in more detail below include (1) Tissue Inhibitors of Matrix Metalloproteinases (TIMPs); (2) tetracyclines, (3) hydroxamates, (4) synthetic MMP fragments (e.g., peptide inhibitors), (5) mercapto-based compounds, and (6) bisphosphonates. Each of these representative examples of classes can, in separate aspects of the invention, be combined with collagen.

1. Tissue Inhibitors of Matrix Metalloproteinase

Tissue Inhibitors of Matrix Metalloproteinases (TIMPs) are classified based upon their ability to inhibit metalloproteinases, structural similarity to each other, the 12 cysteines which form disulfide bonds important in secondary structure, and the presence of a VIRAF motif which interacts with the metal ion of the metalloproteinases. The nucleic acid and amino acid sequences of TIMPs have been described: TIMP-1 (Docherty, A. J. P. et al., (1985) Nature 318: 66-69), TIMP-2 (Boone, T. C., et al. (1990) Proc. Natl. Acad. Sci. 87: 2800-2804; Stetler-Stevenson, W. G, et al. (1990) J. Biol. Chem. 265: 13933-38), and TIMP-3 (Wilde, C. G, et al. (1994) DNA Cell Biol. 13: 711-18; Apte et al., “The Gene Structure of Tissue Inhibitor of Metalloproteinases” (TIMP-3 and Its Inhibitory Activities Define the Distinct TIMP Gene Family); (See also, Boone, T. C., et al., “cDNA cloning and expression of a metalloproteinase inhibitor related to tissue inhibitor of metalloproteinases,” Proc. Natl. Acad. Sci. USA, 87:2800-2804 (Apr. 1990), Freudenstein, mRNA of bovine tissue inhibitor of metalloproteinase: Sequence and expression in bovine ovarian tissue, Biochem Biophys. Res. Comm., 171:250-256 (1990), U.S. Pat. Nos. 5,643,752 and 6,300,310).

TIMP-1 is a 30 kD protein, and is the most commonly expressed TIMP molecule. It contains two asparagine residues which act as carbohydrate binding sites, one in loop 1 and one in loop 2 (Murphy and Docherty, supra). In addition, a truncated form of TIMP-1 that contains only the first three loops of the molecule is able to inhibit MMPs. Although TIMP-1 is a better inhibitor of interstitial collagenase than TIMP-2 (Howard, E. W., et al. (1991) J. Biol. Chem. 266: 13070-75), the 23 kD TIMP-2 molecule is the most effective inhibitor of gelatinases A and B. TIMP-3 is a 21 kD protein which inhibits collagenase 1, stromelysin, and gelatinases A and B (Apte, S. S., et al. (1995) J. Biol. Chem. 270: 14313-18) and may be induced by mitogens (Wick, et al. (1994) J. Biol. Chem. 269: 18953-60).

As described above, any of the four TIMP molecules are capable of inhibiting the activity of virtually all of the MMPs identified to date and would be suitable for the purposes of this invention. However, TIMP-1, which has a high specificity for the inhibition of collagenase, would be particularly preferred for incorporation into a collagen implant.

2. Tetracyclines =p Tetracyclines are a class of analog and derivative compounds known originally for their use as antibiotics. Numerous tetracyclines, including tetracycline, doxycycline, minocycline and others, have been demonstrated to inhibit the production and activity of MMPs. Although the exact mechanism is incompletely understood, MMP inhibition may occur through downregulation of MMP expression and/or post-translationally through chelation of the zinc metal active site. Given their widespread use and low toxicity, these compounds would be of particular utility for incorporation into a collagen implant.

The parent compound of the tetracycline family, tetracycline, has the following general structure:

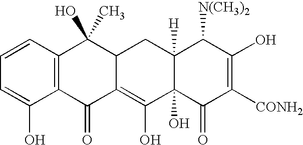

The multiple ring nucleus can be numbered as follows:

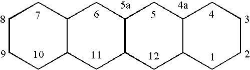

Tetracycline, as well as the 5-OH (oxytetracycline) and 7-Cl (chlorotetracycline) derivatives exist in nature and are well known antibiotics. Other tetracyclines include, for example, apicycline, chelocardin, clomocycline, demeclocycline, doxycycline, etamocycline, guamecycline, lymecycline, meglucyccline, mepycyhcline, minocycline, methacycline, penimepicycline, piacycline, rolitetracycline, and sancycline.

Tetracycylines can also be modified so that they retain their structural relationship to antibiotic tetracyclines, but have their antibiotic activity substantially or completely reduced by chemical modification. Representative examples of chemically modified tetracyclines (CMT's) include, for example, CMT-1 (4-de(dimethylamino)-tetracycline), CMT-2 (tetracyclinonitrile), CMT-3 (6-demethyl-6-deoxy-4-de(dimethylamino)tetracycline), CMT-4 (7-chloro-4-de(dimethylamino)tetra-cycline), CMT-5 (tetracycline pyrazole), CMT-6 (4-hydroxy-4-de(dimethylamino)tetra-cycline), CMT-7 (4-de(dimethylamino)-12α-deoxytetracycline), CMT-8 (6-deoxy-5α-hydroxy-4-de(dimethylamino)tetracycline), CMT-9 (4-de(dimethylamino)-12α-deoxyanhydro-tetracycline), and CMT-10 (4-de(dimethylamino)minocycline).

Representative examples of tetracyclines (including tetracycline derivatives) are described in U.S. Pat. No. 3,622,627 to Blackwood et al., U.S. Pat. No. 3,846,486 to Marcus, U.S. Pat. No. 3,862,225 to Conover et al., U.S. Pat. No. 3,895,033 to Murakami et al., U.S. Pat. No. 3,901,942, to Bernardi et al., U.S. Pat. No. 3,914,299 to Muxfeldt, U.S. Pat. No. 3,925,432 to Gillchriest, U.S. Pat. No. 3,927,094 to Villax, U.S. Pat. No. 3,932,490 to Fernandez, U.S. Pat. No. 3,951,962 to Murakami et al., U.S. Pat. No. 3,983,173 to Hartung et al., U.S. Pat. No. 3,991,111 to Murakami et al., U.S. Pat. No. 3,993,694 to Martin et al., U.S. Pat. No. 4,060,605 to Cotti, U.S. Pat. No. 4,066,694 to Blackwood et al., U.S. Pat. No. 4,081,528 to Armstrong, U.S. Pat. No. 4,086,332 to Armstrong, U.S. Pat. No. 4,126,680 to Armstrong, U.S. Pat. No. 4,853,375 to Krupin et al., U.S. Pat. No. 4,918,208 to Hasegawa et al., and U.S. Pat. No. 5,538,954 to Koch et al. (see generally, Mitscher, L. A., The Chemistry of Tetracycline Antibiotics, ch. 6, Marcell Dekker, New York, 1978).

Further examples of tetracycline derivatives are disclosed in U.S. Patent No. 4,666,897 to Golub et al., U.S. Pat. No. 4,704,383 to McNamara et al., U.S. Pat. No. 4,904,647 to Kulcsar et al., U.S. Pat. No. 4,935,412 to McNamara et al., U.S. Pat. No. 5,223,248 to McNamara et al., U.S. Pat. No. 5,248,797 to Sum et al., U.S. Pat. No. 5,281,628 to Hlavka et al., U.S. Pat. No. 5,326,759 to Hlavka et al., 5,258,371 to Golub et al., U.S. Pat. No. 5,308,839 to Golub et al., U.S. Pat. No. 5,321,017 to Golub et al., U.S. Pat. Nos. 5,326,759 to 5,401,863 to Hlavka et al., U.S. Pat. No. 5,459,135 to Golub et al., U.S. Pat. No. 5,530,117 to Hlvaka et al., 5,563,130 to Backer et al., U.S. Pat. No. 5,567,693 to Backer et al., U.S. Pat. No. 5,574,026 to Backer et al., U.S. Pat. No. 5,698,542 to Zheng et al., U.S. Pat. No. 5,773,430 to Simon et al., U.S. Pat. No. 5,834,450 to Su, U.S. Pat. No. 5,843,925 to Backer et al., 5,856,315 to Backer et al., U.S. Pat. No. 6,028,207 to Zheng et al., U.S. Pat. No. 6,143,161 to Heggie et al. and U.S. Pat. No. 6,165,999 to Vu, as well as PCT publication Nos. WO 99/33455, WO 99/37306, WO 99/37307, WO 00/18353 and WO 00/28983.

3. Hydroxamates

A further class of compounds which inhibit MMPs are hydroxamates (or hydroxamic acids). Although the exact mechanism of MMP inhibition by hydroxamates is not precisely known, it is believed these compounds exert their effect primarily through interaction with the zinc metal active site in the enzyme (e.g., by coordinating with the catalytic zinc in a bidentate manner to adopt a triagonal bipyrimidal geometry). A variety of hydroxamates have been synthesized and tested in several disease states with mixed clinical results. However, given their selective activity against MMPs and their excellent safety and tolerability, these agents would be particularly preferred for incorporation into a collagen implant to enhance the durability of the implant.

Hydroxamates (or hydroxamic acids) have the general structures shown below:

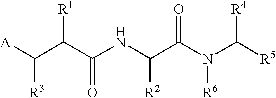

wherein A is HN(OH)—CO— or HCO—N(OH)—; R¹ is C₂-C₅ alkyl; R² is the characterizing group of a natural α amino acid which may be protected provided that R2 is not H or methyl; R³ is H, NH₂, OH, SH, C₁-C₆ alkyl, Cl-C₆ alkoxy, C₁-C₆ alkylamino, C₁-C₆ alkylthio, aryl (C₁-C₆ alkyl), or amino(C₁-C₆ alkyl), hydroxy(C₁-C₆ alkyl), mercapto(C₁-C₆ alkyl) or carboxy(C₁-C₆ alkyl) where the amino, hydroxy, mercapto or carboxyl group can be protected, the amino group may be acylated or the carboxyl group may be amidated; R⁴ is H or methyl; R⁵ is H, C₁-C₆ alkyl, C₁-C₆ alkoxy(C₁-C₆ alkyl), di(C₁-C₆ alkoxy)methylene, carboxy, (C₁-C₆ alkyl)carbonyl, (C₁-C₆ alkoxy)carbonyl, arylmethoxycarbonyl, (C₁-C₆ alkyl)aminocarbonyl or arylaminocarbonyl; and R⁶ is H or methyl; or R² and R⁴ together form a group (CH₂), where n is an integer from 4 to 1; or R⁴ and R⁵ together form a trimethylene group, and pharmaceutically acceptable salts of these hydroxymate compounds that are either acidic or basic. In this regard, see, e.g., EP-A-0236872.

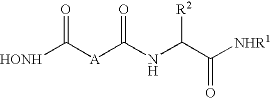

wherein R¹ is C₁-C₆ alkyl; R² is C₁-C₆ alkyl, benzyl, hydroxybenzyl, benzyloxybenzyl, (C₁-C₆ alkoxy) benzyl or benzyloxy (C₁-C₆ alkyl); A is a (CHR³—CHR⁴) or (CR³═CR⁴) group; R³ is hydrogen, C₁-C₆ alkyl, phenyl or phenyl (C₁-C₆ alkyl); and R⁴ is H or C₁-C₆ alkyl, phenyl (C₁-C₆ alkyl), cycloalkyl or cycloalkyl (C₁-C₆ alkyl). In this regard, see, e.g., EP-A-0214639.

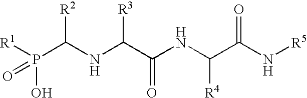

wherein R¹ is hydrogen or hydroxy, R² is hydrogen or alkyl, R³ is C₃-C₆ alkyl, R⁴ is hydrogen, alkyl, —CH₂Z where Z is optionally substituted phenyl or heteroaryl, or R⁴ is a group C(HOR⁸)R⁹ where R⁸ is hydrogen, alkyl of CH₂Ph where Ph is optionally substituted phenyl, and R⁹ is hydrogen or alkyl; and R⁵ is hydrogen or alkyl. In this regard, see, e.g., EP-A-320118.

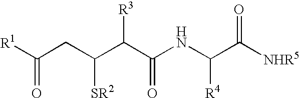

wherein R¹ is hydrogen, alkyl or optionally substituted aryl, R² is hydrogen or acyl such as CO alkyl or COZ where Z is optionally substituted aryl; R³ is C₃₋₆ alkyl, R⁴ is hydrogen, alkyl, —CH₂R¹⁰ where R¹⁰ is optionally substituted phenyl or heteroaryl, or R⁴ is a group C(HOR¹¹)R¹² where R¹¹ is hydrogen, alkyl or CH₂Ph where Ph is optionally substituted phenyl, and R¹² is hydrogen or alkyl; and R⁵ is hydrogen, alkyl or a group C(HR³)COR¹⁴ where R¹³ is hydrogen, or alkyl, and R¹⁴ is hydroxy, alkoxy, or —NR⁶R⁷, where each of R⁶ or R⁷ is hydrogen or alkyl, or R⁶ and R⁷ together with the nitrogen atom to which they are bonded form a 5-, 6 or 7 membered ring with optional oxygen or sulfur atom in the ring or an optional further nitrogen atom optionally substituted by alkyl. In this regard, see, e.g., EP-A-0322184.

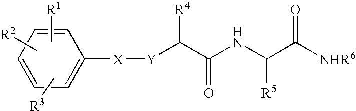

wherein R¹ and R² are independently H, alkyl, alkoxy, halogen or CF₃, R³ is H, acyl, such as COalkyl or COZ, where Z is optionally substituted aryl, or a group RS where R is an organic residue such that the group RS provides an in vivo cleavable disulphide bond; R⁴ is C₃-C₆ alkyl, R⁵ is H, alkyl, —CH₂R¹⁰ where R¹⁰ is optionally substituted phenyl or heteroaryl, or a group C(HOR¹¹)R¹² where R¹¹ is hydrogen, alkyl or CH₂Ph where Ph is optionally substituted phenyl, and R¹² is hydrogen or alkyl; and R⁶ is hydrogen, alkyl or a group C(HR¹³)COR¹⁴ where R¹³ is hydrogen, or alkyl, and R¹⁴ is hydroxy, alkoxy, or —NR⁷R⁸, where each of R⁷ or R⁸ is hydrogen or alkyl, or R⁷ and R⁸ together with the nitrogen atom to which they are bonded form a 5-, 6- or 7-membered ring with optional oxygen, sulfur or optionally substituted nitrogen atom in the ring; or R⁵ and R⁶ are joined together as (CH₂)ₘ where m is an integer from 4 to 12; X is (CH₂), where n is 0, 1, or 2; and Y is CH₂. In this regard, see, e.g., EP-A-358305.

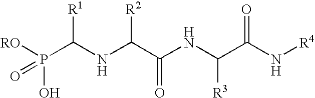

wherein R is hydrogen, C₁-C₆ alkyl or optionally substituted benzyl, R¹ is hydrogen or C₁-C₆ alkyl, R² is C₃-C₆ alkyl, R³ is hydrogen, alkyl, —CH₂Z where Z is optionally substituted phenyl or heteroaryl, or R³ is a group C(HOR⁷)R⁸ where R⁷ is hydrogen, alkyl or CH₂Ph where Ph is optionally substituted phenyl, and R⁸ is hydrogen or alkyl; and R⁴ is —CH₂—(CH₂)ₙOR⁵, —CH₂—(CH₂)ₙOCOR⁶ or —CH(R⁹)COR¹⁰, where n is an integer from 1 to 6; R⁵, R⁶ and R⁹ are hydrogen or C₁-C₆ alkyl; and R¹⁰ is hydroxy or O(C₁-C₆ alkyl) or NR⁵R⁶ where R⁵ and R⁶ may be linked to form a heterocyclic ring; or R³ and R⁴ are joined together as (CH₂)m where m is an integer from 4 to 12. In this regard, see, e.g., EP-A-0401963.

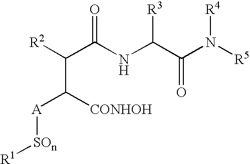

wherein R¹ is H, C₁-C₆ alkyl, phenyl, thienyl, substituted phenyl, phenyl (C₁-C₆)alkyl, heterocyclyl, (C₁-C₆)alkylcarbonyl, phenacyl or substituted phenacyl group; or, when n is 0, R¹ represents SR^{x}, wherein R^{x} represents a group of the formula:

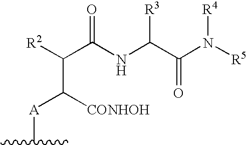

and R² is H, C₁-C₆ alkyl, C₁-C₆ alkenyl, phenyl (C₁-C₆) alkyl, cycloalkyl (C₁-C₆) alkyl or cycloalkenyl (C₁-C₆) alkyl group; R³ is an amino acid side chain or a C₁-C₆ alkyl, benzyl, (C₁-C₆ alkoxy) benzyl, benzyloxy (C₁-C₆ alkyl) or benzyloxybenzyl group; R⁴ is H or a C₁-C₆ alkyl group; R⁵ is H or a methyl group; n is 0, 1 or 2; and A represents a C₁-C₆ hydrocarbon chain, optionally substituted with one or more C₁-C₆ alkyl, phenyl or substituted phenyl groups; and their salts and N-oxides. In this regard, see, e.g., PCT International Publication No. WO90/05719.

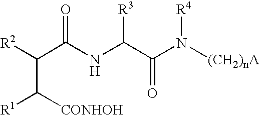

wherein R¹ is H, C₁-C₆ alkyl, C₂-C₆ alkenyl, phenyl, phenyl (C₁-C₆) alkyl, C₁-C₆ alkylthiomethyl, phenylthiomethyl, substituted phenylthiomethyl, phenyl (C₁-C₆) alkylthiomethyl, or heterocyclylthiomethyl or R¹ represents —SR^{x} wherein R^{x} represents a group

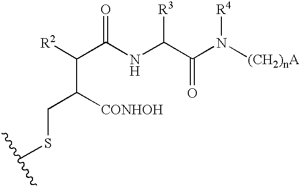

and R² represents a hydrogen atom, or a C₁-C₆ alkyl, C₁-C₆ alkenyl, phenyl (C₁-C₆) alkyl, cycloalkyl (C₁-C₆) alkyl, or cycloalkenyl (C₁-C₆) alkyl; R³ represents an amino acid side chain or a C₁-C₆ alkyl, benzyl, (C₁-C₆) alkoxybenzyl, benzyloxy (C₁-C₆) alkyl, or benzyloxybenzyl group; R⁴ represents a hydrogen atom, or a methyl group; n is an integer from 1 to 6; and A represents the group —NH₂, a substituted acyclic amine or a heterocyclic base; or a salt and/or N-oxide and/or (where the compound is a thio-compound) a sulphoxide or sulphone thereof. In this regard, see, e.g., PCT International Publication No. WO09/05716.

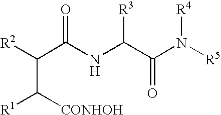

wherein R¹ is H, C₁-C₆ alkyl, C₁-C₆ alkenyl, phenyl, phenyl (C₁-C₆) alkyl, C₁-C₆ alkylthiomethyl, phenylthiomethyl, substituted phenylthiomethyl, phenyl (C₁-C₆) alkylthiomethyl or heterocyclylthiomethyl group; or R¹ represents —S—R^{x}, wherein R^{x} represents a group

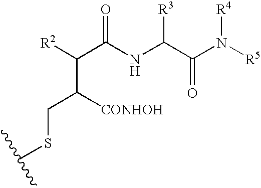

and R² represents a hydrogen atom, or a C₁-C₆ alkyl, C₁-C₆ alkenyl, phenyl (C₁-C₆) alkyl, cycloalkyl (C₁-C₆) alkyl, or cycloalkenyl (C₁-C₆) alkyl; R³ represents an amino acid side chain or a C₁-C₆ alkyl, benzyl, (C₁-C₆) alkoxybenzyl, benzyloxy (C₁-C₆) alkyl or benzyloxybenzyl group; R⁴ represents a hydrogen atom or a methyl group; R⁵ represents a group (CH₂)ₙA; or R⁴ and R⁵ together represent a group

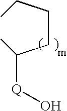

and Q represents CH₂ or CO; m is an integer from 1 to 3; n is an integer from 1 to 6; and A represents a hydroxy, (C₁-C₆) alkoxy, (C₂-C₇) acyloxy, (C₁-C₆) alkylthio, phenylthio, (C₂-C₇) acylamino or N-pyrrolidone group; or a salt and/or N-oxide and/or (where the compound is a thio-compound) a sulphoxide or sulphone thereof. In this regard, see, e.g., PCT International Publication No. WO91/02716.

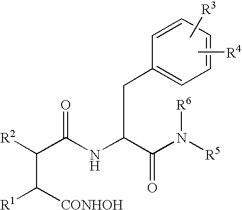

wherein R¹ is H, C₁-C₆ alkyl, phenyl, substituted phenyl, phenyl (C₁-C₆ alkyl), or heterocyclyl; or R¹ is ASOₙR⁷ wherein A represents a C₁-C₆ hydrocarbon chain, optionally substituted with one or more C₁-C₆ alkyl, phenyl or substituted phenyl groups, n is 0, 1, or 2, and R⁷ is C₁-C₆ alkyl, phenyl, substituted phenyl, phenyl (C₁-C₆ alkyl), heterocyclyl, (C₁-C₆ alkyl) acyl, thienyl or phenacyl; R² is hydrogen, C₁-C₆ alkyl, C₁-C₆ alkenyl, phenyl (C₁-C₆ alkyl) or cycloalkyl (C₁-C₆ alkyl); R³ and R⁴ are selected from hydrogen, halogen, cyano amino, amino (C₁-C₆) alkyl, amino di (C₁-C₆) alkyl, amino (C₁-C₆) alkylacyl, aminophenacyl, amino (substituted) phenacyl, amino acid or derivative thereof, hydroxy, oxy (C₁-C₆) alkyl, oxyacyl, formyl, carboxylic acid, carboxamide, carboxy (C₁-C₆) alkylamide, carboxyphenylamide, carboxy (C₁-C₆) alkyl, hydroxy (C₁-C₆) alkyl, (C₁-C₆) alkyloxy (C₁-C₆) alkyl or acyloxy (C₁-C₆) alkyl, (C₁-C₆) alkylcarboxylic acid, or (C₁-C₆) alkylcarboxy (C₁-C₆) alkyl; or R³ is OCH₂COR⁸ and R⁴ is hydrogen wherein R⁸ is hydroxyl, C₁-C₆ oxyalkyl, C₁-C₆ oxyalkylphenyl, amino, C₁-C₆ aminoalkyl, C₁-C₆ aminodialkyl, C₁-C₆ aminoalkylphenyl, an amino acid or derivative thereof; or R³ is OCH₂CH₂OR⁹ and R⁴ is hydrogen wherein R⁹ is C₁-C₆ alkyl, C₁-C₆ alkylphenyl, phenyl, substituted phenyl, (C₁-C₆ alkyl)acyl, or phenacyl; or R³ is OCH₂CN and R⁴ is hydrogen; R⁵ is hydrogen or C₁-C₆ alkyl, or (C₁-C₆) alkylphenyl; R⁶ is hydrogen or methyl; or a salt thereof. In this regard, see, e.g., PCT International Application No. PCT/GB92/00230.

Two preferred compounds for use in the present invention, which are mentioned in U.S. Pat. No. 5,872,152, are: [4-(N-hydroxyamino)-2R-isobutyl-3S-thienylthiomethyl)succinyl]-L-phenylalanine-N-methylamide, having the structure below

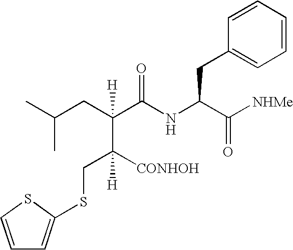

and [4-(N-hydroxyamino)-2R-isobutyl-3S-phenylthiomethyl)succinyl]-L-phenylalanine-N-methylamide, having the structure below

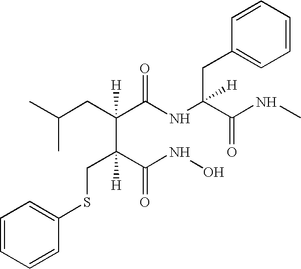

As used herein for describing MMP inhibitors having a hydroxamic acid moiety, the following terms have the indicated meanings. The term “C₁-C₆ alkyl” refers to straight chain or branched chain hydrocarbon groups having from one to six carbon atoms, where illustrative alkyl groups are methyl, ethyl, propyl, isopropyl, butyl, isobutyl, sec-butyl, tert-butyl, pentyl, neopentyl and hexyl. The term “C₁-C₆ alkenyl” refers to straight chain or branched chain hydrocarbon groups having from one to six carbon atoms and having in addition one or more double bonds, each of either E or Z stereochemistry where applicable, where this term would include for example, an alpha, beta-unsaturated methylene, vinyl, 1-propenyl, 1- and 2-butenyl and 2-methyl-2-propenyl, and where in a preferred embodiment the C₁-C₆ alkenyl group is a C₂-C₆ alkenyl group. The term “C₃-C₆ cycloalkyl” refers to an alicyclic group having from 3 to 6 carbon atoms, where illustrative cycloalkyl groups are cyclopropyl, cyclobutyl, cyclopentyl and cyclohexyl. The term “C₄-C₆ cycloalkenyl” refers to an alicyclic group having from 4 to 6 carbon atoms and having in addition one or more double bonds, where illustrative cycloalkenyl groups are cyclopentenyl, cyclohexenyl, cycloheptenyl and cyclooctenyl. The term “halogen” refers to fluorine, chlorine, bromine or iodine. The term “amino acid side chain” refers to a characteristic side chain attached to the —CH(NH₂)(COOH) moiety in the following R or S amino acids: glycine, alanine, valine, leucine, isoleucine, phenylalanine, tyrosine, tryptophan, serine, threonine, cysteine (or cystine), methionine, asparagine, glutamine, lysine, histidine, arginine, glutamic acid and aspartic acid.

Representative examples of hydroxamates, and methods for synthesizing hydroxamates are described in detail in U.S. Pat. Nos. 4,599,361, 4,720,486, 4,743,587, 4,996,358, 5,183,900, 5,189,178, 5,239,078, 5,240,958, 5,256,657, 5,300,674, 5,304,604, 5,310,763, 5,412,145, 5,442,110, 5,473,100, 5,514,677, 5,530,161, 5,643,964, 5,652,262, 5,691,382, 5,696,082, 5,700,838, 5,747,514 5,594,006, 5,763,621, 5,821,262, 5,840,939, 5,849,951, 5,859,253, 5,861,436, 5,866,717, 5,872,152, 5,902,791, 5,917,090, 5,919,940, 5,932,695, 5,962,521, 5,962,529, 6,017,889, 6,022,898, 6,028,110, 6,093,798, 6,103,739, 6,124,329, 6,124,332, 6,124,333 6,127,427, 6,218,389, 6,228,988, and 6,258,851. Representative foreign and international applications and publications include EP-A-0231081, EP-A-0236872, EP-A-0274453, EP-A-0489577, EP-A-0489579, EP-A-0497192, EP-A-0574758, and EP-A-0575844, as well as WO 90/05716, WO 90/05719, WO 91/02716, WO 92/09563, WO 92/17460, WO 92/13831, WO 92/22523, WO 93/09090, WO 93/09097, WO 93/20047, WO 93/24449, WO 93/24475, WO 94/02446, WO 94/02447, WO 94/21612, WO 94/21625, WO 94/24140, WO 94/25434, WO 94/25435, and WO 99/06361. Many hydroxamates are also readily available from a variety of commercial sources.

4. Polypeptide Inhibitors

Within other aspects of the invention polypeptide (including polypeptide derivative) inhibitors of matrix metalloproteinases can be utilized to extend the duration and utility of collagen. Representative examples of polypeptide inhibitors include those disclosed in U.S. Pat. Nos. 5,300,501, 5,530,128, 5,569,665, 5,714,491, and 5,889,058.

5. Mercapto-Based Compounds

Mercapto-based compounds can also be utilized as MMPIs. Representative examples include mercaptoketon and mercaptoalcohol compounds such as those described in U.S. Pat. Nos. 5,831,004, 5,840,698, and 5,929,278; and mercaptosulfides, such as those described in U.S. Pat. No. 5,455,262.

6. Bisphosphonates

Bisphosphonates are compounds which are related to inorganic pyrophosphonic acid (see generally H. Fleisch, Endocr Rev., 19(1):80-100 (1998); see also, H. Fleisch, Bisphosphonates in Bone Disease: From the Laboratory to the Patient (1997, 3rd ed.). The Parthenon Publishing Group, New York and London). Generally, bisphosphonates have the structure: P—C—P. Particularly preferred bisphosphonates have the structure

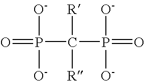

wherein the substituents R′ and R″ independently stand for a hydrogen or a halogen atom, a hydroxy, optionally substituted amino or optionally substituted thio group or an optionally substituted hydrocarbon residue. In one aspect, one of R′ and R″ is hydroxy, hydrogen or chlorine.

Representative examples of bisphosphonates include, for example, alendronate ((4-amino-1-hydroxybutylidene) bisphosphonic acid); clodronate (dichloromethane bisphosphonic acid); etidronate ((1-hydroxyethylidene) bisphosphonic acid); pamidronate ((3-amino-1-hydroxypropylidene) bisphosphonic acid); risedronate ([-hydroxy-2-(3-pyridinyl)ethylidene]bisphosphonic acid); tiludronate (([(4-chloro-phenyl)thio]-methylene]bisphosphonic acid); zolendronate; [1-hydroxy-3-(methyl-pentyl-amino)-propylidene]bis-phosphonate; (BM21.0955, Boehringer Mannheim ); [(cycloheptylamino) methylene]bisphos-phonate (YM175); 1-hydroxy-3-(1-pyrrolidinyl)-propylidene]bisphosphonate (EB-1053); [1-hydroxy-2-(1 H-imidozol-1-yl)ethylidene]bisphosphonate (CGP 42'446, Novartis AC; Switzerland) and (1-hydroxy-2-imidazo-[1,2-a]pyridin-3-yl-ethylidene) bisphosphonate (YM 529, Yamanouchi Pharmaceutical Co., Ltd., Japan). Representative examples of bisphosphonates are described in U.S. Pat. Nos. 5,652,227 and 5,998,390.

7. Combinations of MMPIs

Within additional embodiments of the invention, more than one MMPI may be utilized (i.e., two or more MMPIs can be used in combination). Synergistic MMPIs include, for example tetracyclines and bisphosphonates (see, e.g., U.S. Pat. Nos. 5,998,390 and 6,114,316). Other combinations of MMPIs can likewise be utilized, including for example, MMPIs which inhibit MMPs at different stages (e.g., hydroxamates and tetracyclines).

III. Formulations

As noted above, collagen is a fibrous protein that can be obtained from natural sources or produced recombinantly. Representative examples of U.S. Patents which described collagen-based compositions and methods of preparing such compositions include U.S. Pat. Nos. 6,166,130, 6,051,648, 5,874,500, 5,705,488, 5,550,187, 5,527,856, 5,523,291, 4,582,640, 4,424,208, and 3,949,073.

The MMPI compositions of the present invention can be prepared in a variety of ways. For example, the MMPI can be dissolved directly into the collagen solution. If the MMPI is stable in the collagen solution, the composition containing the collagen and the MMPI can be prepared in a single application apparatus. If the MMPI is not stable in the collagen solution for a significant length of time, the composition can be made as a two-component system in which the components are mixed immediately prior to use.

MMPI compositions of the present invention can also be generated by placing the MMPI in a carrier. Representative examples of carriers include both polymeric and non-polymeric carriers (e.g., liposomes or vitamin-based carriers, which may be either biodegradable or non-biodegradable. Representative examples of biodegradable compositions include albumin, gelatin, starch, cellulose, dextrans, polysaccharides, fibrinogen, poly(esters) [e.g., poly (D,L lactide), poly (D,L-lactide-co-glycolide), poly (glycolide), poly(e-caprolactone), copolymers and blends thereof), poly (hydroxybutyrate), poly (alkylcarbonate), poly(anhydrides) and poly (orthoesters) (see generally, Illum, L., Davids, S. S. (eds.) “Polymers in controlled Drug Delivery” Wright, Bristol, 1987; Arshady, J., Controlled Release 17:1-22 (1991); Pitt, Int. J. Pharm 59:173-196 (1990); Holland et al., J. Controlled Release 4:155-0180 (1986)). Representative examples of nondegradable polymers include copolymers of ethylene oxide and propylene oxide polymers, such as the PLURONIC® polymers available from BASF Corporation (Mount Olive, N.J.), EVA copolymers, silicone rubber, poly(methacrylate) based and poly(acrylate) based polymers. Particularly preferred polymeric carriers include poly (D,L-lactic acid) oligomers and polymers, poly (L-lactic acid) oligomers and polymers, poly (glycolic acid), copolymers of lactic acid and glycolic acid, poly (caprolactone), poly (valerolactone), polyanhydrides, copolymers of caprolactone and/or lactic acid, and/or glycolic acid with polyethylene glycol or methoxypolyethylene glycol and blends thereof.

Polymeric carriers may be fashioned in a variety of forms, including for example, rod-shaped devices, pellets, slabs, or capsules (see, e.g., Goodell et al., Am. J. Hosp. Pharm. 43:1454-1461 (1986); Langer et al., “Controlled release of macromolecules from polymers”; in Biomedical polymers, Polymeric materials and pharmaceuticals for biomedical use, Goldberg, E. P., Nakagim, A. (eds.) Academic Press, pp. 113-137, 1980; Rhine et al., J. Pharm. Sci. 69:265-270 (1980); Brown et al., J. Pharm. Sci. 72:1181-1185 (1983); and Bawa et al., J. Controlled Release 1:259-267 (1985)). An MMPI may be linked by occlusion in the matrices of the polymer, bound by covalent linkages, or encapsulated in microcapsules. Within certain preferred embodiments of the invention, MMPI compositions are provided in non-capsular formulations such as microspheres (ranging from nanometers to micrometers in size), pastes, threads of various size, films and sprays.

Preferably, MMPI compositions of the present invention (which, within certain embodiments comprise one or more MMPI factors, and a polymeric carrier) are fashioned in a manner appropriate to the intended use. Within certain aspects of the present invention, the MMPI composition should be biocompatible, and release one or more MMPI factors over a period of several days to months. For example, “quick release” or “burst” MMPI compositions are provided that release greater than 10%, 20%, or 25% of an MMPI factor (e.g., tetracycline) over a period of 7 to 10 days. Such “quick release” compositions should, within certain embodiments, be capable of releasing chemotherapeutic levels (where applicable) of a desired MMPI factor. Within other embodiments, “low release” MMPI compositions are provided that release less than 5% (w/v) of an MMPI factor over a period of 7 to 10 days. Further, MMPI compositions of the present invention should preferably be stable for several months and capable of being produced and maintained under sterile conditions.

Within certain aspects of the present invention, MMPI compositions may be fashioned in any size ranging from about 0.050 nm to about 500 μm, depending upon the particular use. For example, when used for the purpose of cosmetic tissue augmentation (as discussed below), it is generally preferable to fashion the MMPI composition in microspheres of between about 0.1 to about 100 μm, preferably between about 0.5 and about 50 μm, and most preferably, between about 1 and about 25 μm. Alternatively, such compositions may also be applied as a solution in which the MMPI is solubilized in a micelle. The composition of the micelles may be polymeric in nature. For example, polymeric micelles may include a copolymer of MePEG and poly(D,L-lactide). Alternatively, such compositions may also be applied as a solution in which the MMPI is encapsulated in a liposome (see above). Alternatively, such compositions may also be applied as a solution in which the MMPI is encapsulated in the oil phase of an emulsion or microemulsion.

MMPI compositions of the present invention may also be prepared in a variety of “paste” or gel forms. For example, within one embodiment of the invention, MMPI compositions are provided which are liquid at one temperature (e.g., temperature greater than 37° C., such as 40° C., 45° C., 50° C., 55° C. or 60° C)., and solid or semi-solid at another temperature (e.g., ambient body temperature, or any temperature lower than 37° C.). Such “thermopastes” may be readily made given the disclosure provided herein.

Representative examples of the incorporation of MMPI factors, such as those described above, into a polymeric carriers is described in more detail below in the Examples.

Within further aspects of the present invention, polymeric carriers are provided which are adapted to contain and release a hydrophobic compound, the carrier containing the hydrophobic compound in combination with a carbohydrate, protein or polypeptide. Within certain embodiments, the polymeric carrier contains or comprises regions, pockets, or granules of one or more hydrophobic compounds. For example, within one embodiment of the invention, hydrophobic compounds may be incorporated within a matrix that contains the hydrophobic compound, followed by incorporation of the matrix within the polymeric carrier. A variety of matrices can be utilized in this regard, including for example, carbohydrates and polysaccharides such as starch, cellulose, dextran, methylcellulose, and hyaluronic acid, proteins or polypeptides such as albumin, collagen and gelatin. Within alternative embodiments, hydrophobic compounds may be contained within a hydrophobic core, and this core contained within a hydrophilic shell. For example, as described below in the Examples, paclitaxel may be incorporated into a hydrophobic core (e.g., of the poly D,L lactic acid-PEG or MePEG aggregate) which has a hydrophilic shell.

1. Collagen—MMP Prodrugs

Within certain aspects of the present invention, MMPI compositions may be fashioned in such a manner that the MMPI is covalently attached to the collagen used in the specific application. The MMPI can be attached directly to the collagen or through a linker molecule (e.g., poly(ethylene glycol)). Once the conjugate (i.e., prodrug) is introduced or applied to the desired site, the MMPI may inhibit the MMP while still attached to the collagen or it may inhibit the MMP after it has been cleaved (hydrolytic and/or enzymatic cleavage) from the collagen.

For the TIMPs, a heterobifunctional crosslinking agent (e.g., Sulfo-EMCS [Pierce Chemical Co., Rockford, Ill.]) can be used to covalently bond the TIMP to the collagen. More specifically, the TIMP can be reacted with Sulfo-EMCS such that the maleimide group reacts with the —SH group of the cysteine contained with in the TIMP sequence. The activated TIMP can then be reacted with a collagen solution. The collagen-TIMP conjugate can then be used for tissue augmentation applications.

2. Further Compositions

Within certain embodiments of the invention, the compositions provided herein may be further modified in order to enhance their utility. For example, within one embodiment, a dye or other coloring agent may be added to enhance visualization of the composition. The dye or coloring agent may be either permanent or transient (e.g., methylene blue). Within other embodiments, compounds or factors which aid clotting (e.g., thrombin) may be added to the compositions described herein.

With yet other embodiments, the compositions provided herein may further include additional compounds or agents that encourage or stimulate bone growth, including for example, hydroxyapatite and/or bone morphogenic proteins (e.g., BMP-1 to BMP-9), which are described, for example, in U.S. Pat. Nos. 4,877,864; 5,013,649; 5,661,007; 5,688,678; 6,177,406; 6,432,919; and 6,534,268; and Wozney, J. M., et al., Science: 242(4885): 1528-1534 (1988).

IV. Clincal Application

1. MMPI-Loaded Collagen-Based Orthopedic Implants

A variety of collagen implants have been developed for use in orthopedic surgery as a substitute for autogenous or allogenous bone grafts. Collagen is the principle organic component of bone and can be combined with mineral formulations, autogenous bone marrow, bone graft, and/or growth factors (such as BMPs) for use as a bone substitute or a skeletal repair product. Typical applications include, but are not restricted to, total joint replacement surgery (e.g. artificial hips, knees, etc.), spinal fusion surgery, long bone fractures, repair of traumatic bone defects, voids, or gaps, to augment an autograph, and as a bone filler at bone graft harvesting sites. Examples of commercially available collagen-based bone grafts include COLLAGRAFT Paste and COLLAGRAFT Strips made by Neucoll, Inc. (Campbell, Calif.). COLLAGRAFT is a combination of highly purified Type I bovine dermal fibrillar collagen and a mixture of 65% hydroxyapatite and 35% tricalcium phosphate. This material closely resembles human bone and is resorbed and replaced with bone during the healing process. Representative examples of bone grafts are described in U.S. Pat. Nos. 6,083,522 and 6,280,474, and in PCT publication No. WO 98/52498.

In one aspect of the present invention, an MMPI is added to the collagen matrix in a sustained-release form to decrease the rate of degradation of the bone graft material and prolong its activity in vivo beyond that seen with collagen alone. This allows the matrix to function as a scaffold for longer periods of time allowing stronger, more mature bone growth to occur prior to dissolution of the collagen matrix. Any MMPI described above could be utilized alone, or in combination, in the practice of this embodiment. Preferred MMPI's for use in bone grafts include TIMP-1, tetracycline, doxycycline, minocycline, and other chemically-modified tetracyclines (CMTs), BATIMISTAT, MARIMISTAT, RO-1130830, CGS 27023A, BMS-275291, CMT-3, SOLIMASTAT, ILOMASTAT, CP-544439, PRINOMASTAT, PNU-1427690, SU-5402 and TROCADE, as well as analogues and derivatives of the aforementioned. All of these agents are suitable for use in combination with factors that encourage bone growth including, but not restricted to, BMPs (e.g. BMP-2), autogenous marrow, mineral, and autologous bone graft material. The following particularly preferred compositions are ideally suited for use in this indication.

a. MARIMASTAT-Loaded Collagen Bone Graft Matrix

The preferred MARIMASTAT-loaded collagen bone graft matrix is about 0.001% -30% MARIMASTAT by weight (i.e., 1 μg-30mg MARIMASTAT per 100 mg of collagen implant). A particularly preferred dosage is 0.01-15% MARIMASTAT by weight (i.e., 10 μg-15 mg per 100 mg of collagen paste). Alternatively, since the material is often packaged as a strip, drug dosage can also be determined as a function of area. Preferred dosing of MARIMASTAT using this dosing regimen is 1 μg-37.5 μg/mm² of collagen strip. The total dosage delivered in a MARIMASTAT-loaded collagen orthopedic implant procedure would typically not exceed 45 mg (or less than the established well tolerated single daily does of 50 mg). In one embodiment, 0.001-30% MARIMASTAT by weight is loaded into PLGA microspheres, which are in turn loaded into the collagen implant, to produce sustained release of the drug over a period ranging from several days to several months. Any source of collagen (e.g., porcine, bovine, human, or recombinant; crosslinked or noncrosslinked) is suitable to be combined with the above to produce the desired end product. It should also be readily evident to one of skill in the art that pharmaceutically acceptable analogues and derivatives of MARIMASTAT are also suitable for use in this embodiment either alone or in combination with other MMPIs.

b. BATIMASTAT-Loaded Collagen Bone Graft Matrix

The preferred BATIMASTAT-loaded collagen bone graft matrix is 0.001-30% BATIMASTAT by weight (i.e., 1 ug-30 mg BATIMASTAT per 100 mg of collagen implant). A particularly preferred dosage is 0.01 to 30% by weight (10 μg-30 mg per 100 mg of collagen paste). Alternatively, since the material is often packaged as a strip, drug dosage can also be determined as a function of area. Preferred dosing of BATIMASTAT using this dosing regimen is 1 μg-200 μg/mm² of collagen strip. Regardless, the total dosage delivered in a BATIMASTAT-loaded collagen orthopedic implant procedure would not exceed 240 mg of BATIMASTAT (or less than the established well tolerated single dose of 300 mg/m2). In one embodiment, 0.001-30% BATIMASTAT by weight is loaded into PLGA microspheres, which are in turn loaded into the collagen implant, to produce sustained release of the drug over a period ranging from several days to several months. Any source of collagen (e.g., porcine, bovine, human, or recombinant; crosslinked or non-crosslinked) is suitable to be combined with the above to produce the desired end product. Pharmaceutically acceptable analogues and derivatives of BATIMASTAT are also suitable for use in this embodiment either alone or in combination with other MMPIs.

c. Doxycycline-Loaded Collagen Bone Graft Matrix

The preferred doxycycline-loaded collagen bone graft matrix is 0.001-30% doxycycline by weight (1 ug-30 mg doxycycline per 100 mg of collagen implant). A particularly preferred dosage is 0.01-30% doxycycline by weight (10 μg-30 mg doxycycline per 100 mg of collagen paste). Alternatively, since the material is often packaged as a strip, drug dosage can also be determined as a function of area. Preferred dosing of doxycycline using this dosing regimen is 1μg-83 μg/mm² of collagen strip. The total dosage delivered in a doxycycline-loaded collagen orthopedic implant procedure should typically not exceed 150 mg of doxycycline (or less than the established well tolerated single dose of 200 mg). In one embodiment, 0.01-30% doxycycline by weight is loaded into PLGA microspheres, which are in turn loaded into the collagen implant, to produce sustained release of the drug over a period ranging from several days to several months. Any source of collagen (e.g., porcine, bovine, human, or recombinant; crosslinked or non-crosslinked) is suitable to be combined with the above to produce the desired end product. Pharmaceutically acceptable analogues and derivatives of doxycycline are also suitable for use in this embodiment either alone or in combination with other MMPIs.

d. Tetracycline-Loaded Collagen Bone Graft Matrix

A preferred tetracycline-loaded collagen bone graft matrix is 0.001-30% tetracycline by weight (1 ug-30 mg tetracycline per 100 mg of collagen implant). A particularly preferred dosage is 0.01-30% tetracycline by weight (10 μg-30 mg tetracycline per 100 mg of collagen paste). Alternatively, since the material is often packaged as a strip, drug dosage can also be determined as a function of area. Preferred dosing of tetracycline using this dosing regimen is 1 μg-625 μg/mm² of collagen strip. The total dosage delivered in a tetracycline-loaded collagen orthopedic implant procedure should typically not exceed 750 mg of tetracycline (or less than the established well tolerated single dose of 1000 mg). In one embodiment, 0.001-30% tetracycline by weight is loaded into PLGA microspheres, which are in turn loaded into the collagen implant, to produce sustained release of the drug over a period ranging from several days to several months. Any source of collagen (e.g., porcine, bovine, human, or recombinant; crosslinked or non-crosslinked) is suitable to be combined with the above to produce the desired end product. Pharmaceutically acceptable analogues and derivatives of tetracycline, including chemically-modified tetracylines (CMTs), are also suitable for use in this embodiment either alone or in combination with other MMPIs.

e. Minocycline-Loaded Collagen Bone Graft Matrix

A preferred minocycline-loaded collagen bone graft matrix is 0.001-30% minocycline by weight (1 μg-30 mg minocycline per 100 mg of collagen implant). A particularly preferred dosage is 0.01-6% minocycline by weight (10 μg-6 mg minocycline per 100 mg of collagen paste). Alternatively, since the material is often packaged as a strip, drug dosage can also be determined as a function of area. Preferred dosing of minocycline using this dosing regimen is 1 μg-150 μg/mm² of collagen strip. The total dosage delivered in a minocyline-loaded collagen orthopedic implant procedure should typically not exceed 180 mg of minocycline (or less than the established tolerated single dose of 200 mg). In one embodiment, 0.001-30% minocycline is loaded into PLGA microspheres, which are in turn loaded into the collagen implant, to produce sustained release of the agent over a period ranging from several days to several months. Any source of collagen (e.g., porcine, bovine, human, or recombinant; crosslinked or non-crosslinked) is suitable to be combined with the above to produce the desired end product. Pharmaceutically acceptable analogues and derivatives of minocycline are also suitable for use in this embodiment either alone or in combination with other MMPIs.

f. TROCADE-Loaded Collagen Bone Graft Matrix

A preferred TROCADE-loaded collagen bone graft matrix is 0.001-30% TROCADE by weight (1 μg-30 mg TROCADE per 100 mg of collagen implant). A particularly preferred dosage is 0.01 to 5% TROCADE by weight (10 μg-5 mg TROCADE per 100 mg of collagen paste). Alternatively, since the material is often packaged as a strip, drug dosage can also be determined as a function of area. Preferred dosing of TROCADE using this dosing regimen is 1 μg-100 μg/mm² of collagen strip. The total dosage delivered in a TROCADE-loaded collagen orthopedic implant procedure should typically not exceed 120 mg of TROCADE (or less than the established well tolerated single dose of 150 mg). In one embodiment, 0.001-30% TROCADE by weight is loaded into PLGA microspheres, which are in turn loaded into the collagen implant, to produce sustained release of the drug over a period ranging from several days to several months. Any source of collagen (e.g., porcine, bovine, human, or recombinant; crosslinked or non-crosslinked) is suitable to be combined with the above to produce the desired end product. Pharmaceutically acceptable analogues and derivatives of TROCADE are also suitable for use in this embodiment either alone or in combination with other MMPIs.

2. MMPI-Loaded Collagen Containing Spinal Fusion Devices

Implantable medical devices containing collagen sponges have been developed to improve the outcome of spinal fusion surgery. When conservative management of degenerative disc disease is ineffective, it often becomes necessary to surgically fuse together the adjacent bony lumbar segments on either side of an affected disc. An example of a collagen-containing medical device used in spinal fusion surgery is the LT-CAGE and INFUSE Bone Graft system developed by Medtronic Sofamor Danek, Inc. (Memphis, Tenn.). The LT-CAGE system is a threaded metallic cylinder with a hollow core that is placed across the diseased disc and anchored into the vertebrae above and below it. Using an anterior approach (in either open surgery or laparoscopic surgery), the surgeon accesses the spine and removes a portion of the degenerated disc from the affected disc space. The metal cage is then placed into the disc space to provide support and restore normal anatomic positioning to the spine until bone fusion occurs. The hollow core of the cage allows the placement of materials, such as autologous bone grafts and bone morphogenic proteins (BMPs), that will encourage bone ingrowth. Representative examples of suitable spinal fusion devices are described in U.S. Pat. Nos. 5,702, 449 and 5,645,084.

Type I bovine absorbable collagen sponge is used as a carrier for the INFUSE recombinant bone morphogenic protein-2 (BMP-2) (available from Medtronic Sofamor Danek). The collagen sponge is hydrated with a solution containing BMP-2, rolled up and placed into the cage prior to its placement into the disc space. Once in place, the BMP is slowly released from the collagen matrix to stimulate bone growth, while the matrix itself acts as a scaffold for the deposition of new bone. In the present invention, an MMPI is added to a collagen sponge in a sustained-release form to decrease the rate of degradation of the implant and prolong its activity in vivo beyond that seen with collagen alone. This would allow the matrix to function as a scaffold for longer periods of time allowing stronger, more mature bone growth to occur prior to dissolution of the collagen matrix.

Any MMPI described previously could be utilized alone, or in combination, in the practice of this embodiment. Representative MMPI's for use in spinal implants include TIMP. 1, tetracycline, doxycycline, minocycline, and other chemically-modified tetracyclines (CMTs), BATIMASTAT, MARIMASTAT, RO-1130830, CGS 27023A, BMS-275291, CMT-3, SOLIMASTAT, ILOMASTAT, CP-544439, PRINOMASTAT, PNU-1427690, SU-5402 and TROCADE, as well as analogues and derivatives of the aforementioned. All of these agents are suitable for use in combination with factors that encourage bone growth including, but not restricted to, BMPs (e.g. BMP-2 or BMP-8) and autologous bone graft material. The following particularly preferred compositions are ideally suited for use in this indication:

a. MARIMASTAT-Loaded Collagen Spinal Implants

A preferred MARIMASTAT-loaded spinal collagen implant is 0.001% -30% MARIMASTAT by weight (i.e., 1 μg-30 mg MARIMASTAT per 100 mg of collagen implant). A particularly preferred dosage is 0.01-15% MARIMASTAT by weight (i.e., 10 μg-15 mg per 100 mg of collagen implant). Alternatively, since the material is often packaged as a sheet drug dosage can also be determined as a function of area. Preferred dosing of MARIMASTAT using this dosing regimen is 1 μg-37.5 μg/mm³ of collagen implant. The total dosage delivered in spinal fusion treatment should typically not exceed 45 mg (or less than the established well tolerated single daily does of 50 mg). In one embodiment, 0.001-30% MARIMASTAT by weight is loaded into PLGA microspheres, which are in turn loaded into the collagen implant, to produce sustained release of the drug over a period ranging from several days to several months. Any source of collagen (e.g., porcine, bovine, human, or recombinant; crosslinked or non-crosslinked) is suitable to be combined with the above to produce the desired end product. Pharmaceutically acceptable analogues and derivatives of MARIMASTAT are also suitable for use in this embodiment either alone or in combination with other MMPIs.

b. BATIMASTAT-Loaded Collagen Spinal Implants

A preferred composition is 0.001-30% BATIMASTAT by weight (i.e., 1 μg-30 mg BATIMASTAT per 100 mg of collagen implant). A particularly preferred dosage is 0.01 to 30% by weight (10 μg-30 mg per 100 mg of collagen implant). Alternatively, since the material is often packaged as a sheet, drug dosage can also be determined as a function of area. Preferred dosing of BATIMASTAT using this dosing regimen is 1 μg-200 μg/mm³ of collagen implant. The total dosage delivered in a BATIMASTAT-loaded collagen spinal implant should typically not exceed 240 mg of BATIMASTAT (or less than the established well tolerated single dose of 300 mg/²). In one embodiment, 0.001-30% BATIMASTAT by weight is loaded into PLGA microspheres, which are in turn loaded into the collagen implant, to produce sustained release of the drug over a period ranging from several days to several months. Any source of collagen (e.g., porcine, bovine, human, or recombinant; crosslinked or non-crosslinked) is suitable to be combined with the above to produce the desired end product. Pharmaceutically acceptable analogues and derivatives of BATIMASTAT are also suitable for use in this embodiment either alone or in combination with other MMPIs.

c. Doxycycline-Loaded Collagen Spinal Implants.

A preferred composition is 0.001-30% doxycycline by weight (1 μg-30 mg doxycycline per 100 mg of collagen implant). A particularly preferred dosage is 0.01-30% doxcycline by weight (10 μg-30 mg doxycycline per 100 mg of collagen implant). Alternatively, since the material is often packaged as a sheet, drug dosage can also be determined as a function of area. Preferred dosing of doxycycline using this dosing regimen is 1 μg-83 μg/mm³ of collagen implant. The total dosage delivered in a doxycycline-loaded collagen spinal implant should typically not exceed 100 mg of doxycycline (or less than the established well tolerated single dose of 200 mg). In one embodiment, 0.001-30% doxycycline by weight is loaded into PLGA microspheres, which are in turn loaded into the collagen implant, to produce sustained release of the drug over a period ranging from several days to several months. Any source of collagen (e.g., porcine, bovine, human, or recombinant; crosslinked or non-crosslinked) is suitable to be combined with the above to produce the desired end product. Pharmaceutically acceptable analogues and derivatives of doxycycline are also suitable for use in this embodiment either alone or in combination with other MMPIs.

d. Tetracycline-Loaded Collagen Spinal Implants

A preferred composition is 0.001-30% tetracycline by weight (1 μg-30 mg tetracycline per 100 mg of collagen implant). A particularly preferred dosage is 0.01-30% tetracycline by weight (10 μg-30 mg tetracycline per 100 mg of collagen implant). Alternatively, since the material is often packaged as a sheet, drug dosage can also be determined as a function of area. Preferred dosing of tetracycline using this dosing regimen is 1 μg-625 μg/mm³ of collagen implant. The total dosage delivered in a tetracycline-loaded collagen spinal implant should typically not exceed 750 mg of tetracycline (or less than the established well tolerated single dose of 1000 mg). In one embodiment, 0.001-30% tetracycline by weight is loaded into PLGA microspheres, which are in turn loaded into the collagen implant, to produce sustained release of the drug over a period ranging from several days to several months. Any source of collagen (e.g., porcine, bovine, human, or recombinant; crosslinked or non-crosslinked) is suitable to be combined with the above to produce the desired end product. Pharmaceutically acceptable analogues and derivatives of tetracycline, including chemically-modified tetracylines (CMTs), are also suitable for use in this embodiment either alone or in combination with other MMPIs.

e. Minocycline-Loaded Collagen Spinal Implants

A preferred composition is 0.001-30% minocycline by weight (1 μg-30 mg minocycline per 100 mg of collagen implant). A particularly preferred dosage is 0.01-6% minocycline by weight (10 μg-6 mg minocycline per 100 mg of collagen implant). Alternatively, since the material is often packaged as a sheet, drug dosage can also be determined as a function of area. Preferred dosing of minocycline using this dosing regimen is 1 μg-150 μg/mm³ of collagen implant. The total dosage delivered in a collagen spinal implant should typically not exceed 180 mg of minocycline (or less than the established tolerated single dose of 200 mg). In one embodiment, 0.001-30% minocycline is loaded into PLGA microspheres, which are in turn loaded into the collagen implant, to produce sustained release of the agent over a period ranging from several days to several months. Any source of collagen (e.g., porcine, bovine, human, or recombinant; crosslinked or non-crosslinked) is suitable to be combined with the above to produce the desired end product. Pharmaceutically acceptable analogues and derivatives of minocycline are also suitable for use in this embodiment either alone or in combination with other MMPIs.

f. TROCADE-Loaded Collagen Spinal Implants

A preferred composition is 0.001-30% TROCADE by weight (1 μg-30 mg TROCADE per 100 mg of collagen implant). A particularly preferred dosage is 0.01 to 5% TROCADE by weight (10 μg-5 mg TROCADE per 100 mg of collagen implant). Alternatively, since the material is often packaged as a sheet, drug dosage can also be determined as a function of area. Preferred dosing of TROCADE using this dosing regimen is 1 μg-100μg/mm³ of collagen implant. The total dosage delivered in a TROCADE-loaded collagen spinal implant should typically not exceed 120 mg of TROCADE (or less than the established well tolerated single dose of 150 mg). In one embodiment, 0.001-30% TROCADE by weight is loaded into PLGA microspheres, which are in turn loaded into the collagen implant, to produce sustained release of the drug over a period ranging from several days to several months. Any source of collagen (e.g., porcine, bovine, human, or recombinant; crosslinked or non-crosslinked) is suitable to be combined with the above to produce the desired end product. Pharmaceutically acceptable analogues and derivatives of TROCADE are also suitable for use in this embodiment either alone or in combination with other MMPIs.

3. MMPI-Loaded Collagen Surgical Meshes, Slings and Patches

Several collagen-based surgical meshes have been produced to function as tissue repair products for use during open surgery. Products such as FORTAGEN Surgical Mesh (Organogenesis Inc., Canton, Mass.), GRAFTPATCH (Organogenesis Inc., Canton, Mass.), and SURGISIS (Cook Biotech, Inc., West Lafayette, Ind.) consist of a multilaminate sheet composed primarily of Type I collagen (usually porcine or bovine) that is used to reinforce soft tissues during operative repair. Indications include defects of the abdominal and thoracic wall, muscle flap reinforcement, rectal and vaginal prolapse, repair of tissue flap donor sites, ostomy reinforcement, reconstruction of the pelvic floor, hernia repair, suture line reinforcement and reconstructive purposes.

Surgical slings, such as the FORTAFLEX Surgical Sling (Organogenesis Inc., Canton, Mass.) and the SURGISIS Sling are also composed predominantly of Type I Collagen (usually porcine or bovine) and are utilized in open urological surgery procedures. Indications include pubourethral support, prolapse repair (urethral, vaginal, rectal and colonic), rectoceles, cystoceles, enteroceles, mastoplexy, reconstruction of the pelvic floor, bladder support, sacrocolposuspension and other reconstructive procedures.

Collagen surgical patches are also be used in tendon, ligament and cartilage repair surgeries. Over 700,000 ligament and tendon repairs are performed annually in the United States including: repairs of the foot and ankle (11% of the total—particularly the Achilles tendon; also peroneal tendons, plantar fascia repair, extensor digitorum tendons, anterior tibial tendon, lateral stabilizing ligaments of the ankle, anterior inferior tibial fibular ligament, medial deltoid ligament), knee (38% of the total—particularly the medial collateral ligament, lateral collateral ligament, anterior cruciate ligament, posterior cruciate ligament, meniscal repair; also chondral surface repair, patellar tendon repair, bicep femoris tendon repair), hip (rectus femoris origin, gracilis tendon, avulsion of the hamstring muscle origins), pelvis (gracilis muscle origin, adductor muscle origins, rectus femoris insertion, pubic symphysis cartilage), shoulder (25% of the total—particularly the rotator cuff tendons; also acromioclavicular stabilizing ligaments, biceps tendons), back (sacroiliac stabilizing ligaments), elbow (biceps tendons, lateral epicondyle—extensor origins, medial epicondyle—flexor origins, triceps complex), and hand (26% of the total—flexor and extensor tendons of the wrist and hand). Collagenous patches, such as the FORTAFLEX™ Patch, are used to reinforce the tissue during surgical repair and healing. Tendon and ligament repair surgeries typically involve the use of suture anchors or suture-passing devices to secure the damaged tendons to the bone. Depending on the size of the tear, a collagen patch may be used to fill a defect in the tendon or ligament.

In all the above cases, the collagen implant serves as a resorbable scaffold that provides biomechanical strength, support and reinforcement of soft tissues that are surgically repaired. Eventually the collagen becomes infiltrated and replaced by host tissue cells, which are able to repair and regenerate the damaged tissue. For many of these surgical interventions, durability of the collagen implant becomes an important clinical issue. In urinary procedures, the surgical correction of tissue defects (particularly abdominal wall and hernia repairs) and in tendon and ligament repairs, it is desirable for the collagen implant to provide structural integrity until full healing can occur. In the case of large tissue defects, which can take several months to over a year to heal, limited durability of the collagen implant can become a clinical problem if it completely absorbs prior to the completion of healing.

In an attempt to address this problem, manufacturers have attempted to produce a collagen implant with improved durability through increased collagen crosslinking. Utilizing this process, products such as FORTAPERM surgical implants (Organogenesis Inc., Canton, Mass.) can function as a tissue support for longer periods. However, there remains a need for the production of collagen surgical meshes, slings and patches with sustained structural integrity and slower degradation times. Utilizing an MMPI-loaded collagen-based surgical mesh, sling or patch according to the present invention can sustain the activity of the implant and allow more effective and complete healing of the soft tissue defect. The implant would still ultimately degrade, but last for longer than currently available biodegradable implants. This is also superior to permanent implants, such as e-PTFE surgical meshes, e.g., GORE-TEX (Gore & Associates, Inc., Newark, Del.), which can require a second operative procedure to remove.

Any MMPI described above could be utilized alone, or in combination, in the practice of this embodiment. Preferred MMPIs for use as surgical meshes, slings and patches include TIMP-1, tetracycline, doxycycline, minocycline, and other chemically-modified tetracyclines (CMTs), BATIMASTAT, MARIMASTAT, RO-1-130830, CGS 27023A, BMS-275291, CMT-3, SOLIMASTAT, ILOMASTAT, CP-544439, PRINOMASTAT, PNU-1427690, SU-5402 and TROCADE, as well as analogues and derivatives of the aforementioned. The following particularly preferred compositions are ideally suited for use in this indication:

a. MARIMASTAT-Loaded Collagen Surgical Meshes, Slings and Patches

A preferred MARIMASTAT-loaded collagen surgical mesh, sling or patch is 0.001% -30% MARIMASTAT by weight (i.e., 1 μg-30 mg MARIMASTAT per 100 mg of surgical mesh, sling or patch). A particularly preferred dosage is 0.01-15% MARIMASTAT by weight (i.e., 10 μg-15 mg per 100 mg of collagen implant). Alternatively, since the material is often packaged as a sheet (typical sizes are 2 cm×5 cm; 5 cm×5 cm; 12 cm×36 cm), drug dosage can also be determined as a function of area. Preferred dosing of MARIMASTAT using this dosing regimen is 1 μg-104 μg/cm² of collagen sheet. The total dosage delivered in a soft tissue repair should typically not exceed 45 mg (or less than the established well tolerated single daily does of 50 mg). Regardless of the size or type of collagen implant employed (surgical mesh, sling or patch), the total drug content should typically not exceed 50 mg of MARIMASTAT. In one embodiment, 0.001-30% MARIMASTAT by weight is loaded into PLGA microspheres, which are in turn loaded into the collagen implant, to produce sustained release of the drug over a period ranging from several days to several months. Any source of collagen (e.g., porcine, bovine, human, or recombinant; crosslinked or non-crosslinked) is suitable to be combined with the above to produce the desired end product. Pharmaceutically acceptable analogues and derivatives of MARIMASTAT are also suitable for use in this embodiment either alone or in combination with other MMPIs.

b. BATIMASTAT-Loaded Collagen Surgical Meshes Slings and Patches

A preferred composition is 0.001-30% BATIMASTAT by weight (i.e., 1 μg-30 mg BATIMASTAT per 100 mg of collagen surgical mesh, sling or patch). A particularly preferred dosage is 0.01 to 30% by weight (10 μg-30 mg per 100 mg of collagen surgical mesh, sling or patch). Alternatively, since the material is often packaged as a sheet (typical sizes are 2 cm×5 cm; 5 cm×5 cm; 12 cm×36 cm), drug dosage can also be determined as a function of area. Preferred dosing of BATIMASTAT using this dosing regimen is 1 μg-555 μg/cm² of collagen implant. The total dosage delivered in a 12 cm×36 cm BATIMASTAT-loaded collagen surgical implant should typically not exceed 240 mg of BATIMASTAT (or less than the established well tolerated single dose of 300 mg/m²). Regardless of the size or type of collagen implant employed (surgical mesh, sling or patch), the total drug content should typically not exceed 300 mg of BATIMASTAT. In one embodiment, 0.001-30% BATIMASTAT by weight is loaded into PLGA microspheres, which are in turn loaded into the collagen implant, to produce sustained release of the drug over a period ranging from several days to several months. Any source of collagen (e.g., porcine, bovine, human, or recombinant; crosslinked or non-crosslinked) is suitable to be combined with the above to produce the desired end product. Pharmaceutically acceptable analogues and derivatives of BATIMASTAT are also suitable for use in this embodiment either alone or in combination with other MMPIs.

c. Doxycycline-Loaded Collagen Surgical Meshes, Slings and Patches

A preferred composition is 0.001-30% doxycycline by weight (1 μg-30 mg doxycycline per 100 mg of collagen surgical mesh, sling or patch). A particularly preferred dosage is 0.01-30% doxcycline by weight (10 μg-30 mg doxycycline per 100 mg of collagen surgical mesh, sling or patch). Alternatively, since the material is often packaged as a sheet (typical sizes are 2 cm×5 cm; 5 cm×5 cm; 12 cm×36 cm), drug dosage can also be determined as a function of area. Preferred dosing of doxycycline using this dosing regimen is 1 μg-350 μg/cm2 of collagen implant. The total dosage delivered in a 12 cm×36 cm doxycycline-loaded collagen surgical implant would typically not exceed 160 mg of doxycycline (or less than the established well tolerated single dose of 200 mg). Regardless of the size or type of collagen implant employed (surgical mesh, sling or patch), the total drug content should typically not exceed 200 mg of doxycycline. In one embodiment, 0.001-30% doxycycline by weight is loaded into PLGA microspheres, which are in turn loaded into the collagen implant, to produce sustained release of the drug over a period ranging from several days to several months. Any source of collagen (e.g., porcine, bovine, human, or recombinant; crosslinked or non-crosslinked) is suitable to be combined with the above to produce the desired end product. Pharmaceutically acceptable analogues and derivatives of doxycycline are also suitable for use in this embodiment either alone or in combination with other MMPIs.

d. Tetracycline-Loaded Collagen Surgical Meshes Slings and Patches

A preferred composition is 0.001-30% tetracycline by weight (1 μg-30 mg tetracycline per 100 mg of collagen surgical mesh, sling or patch). A particularly preferred dosage is 0.01-30% tetracycline by weight (10 μg-30 mg tetracycline per 100 mg of collagen surgical mesh, sling or patch). Alternatively, since the material is often packaged as a sheet (typical sizes are 2 cm×5 cm; 5 cm×5 cm; 12 cm×36 cm), drug dosage can also be determined as a function of area. Preferred dosing of tetracycline using this dosing regimen is 1 μg-1.75 mg/cm² of collagen implant. Therefore, the total dosage delivered in a 12 cm×36 cm tetracycline-loaded collagen surgical implant would typically not exceed 760 mg of tetracycline (or less than the established well tolerated single dose of 1000 mg). Regardless of the size or type of collagen implant employed (surgical mesh, sling or patch), the total drug content should typically not exceed 1000 mg of tetracycline. In one embodiment, 0.001-30% tetracycline by weight is loaded into PLGA microspheres, which are in turn loaded into the collagen implant, to produce sustained release of the drug over a period ranging from several days to several months. Any source of collagen (e.g., porcine, bovine, human, or recombinant; crosslinked or non-crosslinked) is suitable to be combined with the above to produce the desired end product. Pharmaceutically acceptable analogues and derivatives of tetracycline, including chemically-modified tetracylines (CMTs), are also suitable for use in this embodiment either alone or in combination with other MMPIs.

e. Minocycline-Loaded Collagen Surgical Meshes, Slings and Patches

A preferred composition is 0.001-30% minocycline by weight (1 μg-30 mg minocycline per 100 mg of collagen surgical mesh, sling or patch). A particularly preferred dosage is 0.01-6% minocycline by weight (10 μg-6 mg minocycline per 100 mg of collagen surgical mesh, sling or patch). Alternatively, since the material is often packaged as a sheet (typical sizes are 2 cm×5 cm; 5 cm×5 cm; 12 cm×36 cm), drug dosage can also be determined as a function of area. Preferred dosing of minocycline using this dosing regimen is 1 μg-415 μg/cm² of collagen implant. The total dosage delivered in a 12 cm×36 cm minocycline-loaded collagen surgical implant would typically not exceed 180 mg of minocycline (or less than the established tolerated single dose of 200 mg). Regardless of the size or type of collagen implant employed (surgical mesh, sling or patch), the total drug content should typically not exceed 200 mg of minocycline. In one embodiment, 0.001% -30% minocycline is loaded into PLGA microspheres, which are in turn loaded into the collagen implant, to produce sustained release of the agent over a period ranging from several days to several months. Any source of collagen (e.g., porcine, bovine, human, or recombinant; crosslinked or non-crosslinked) is suitable to be combined with the above to produce the desired end product. Pharmaceutically acceptable analogues and derivatives of minocycline are also suitable for use in this embodiment either alone or in combination with other MMPIs.

f. TROCADE-Loaded Collagen Surgical Meshes Slings and Patches

A preferred composition is 0.001-30% TROCADE by weight (1 μg-30 mg TROCADE per 100 mg of collagen surgical mesh, sling or patch). A particularly preferred dosage is 0.01 to 5% TROCADE by weight (10 μg-5 mg TROCADE per 100 mg of collagen surgical mesh, sling or patch). Alternatively, since the material is often packaged as a sheet (typical sizes are 2 cm×5 cm; 5 cm×5 cm; 12 cm×36 cm), drug dosage can also be determined as a function of area. Preferred dosing of TROCADE using this dosing regimen is 1 μg-275 μg/cm² of collagen implant. Therefore, the total dosage delivered in a 12 cm×36 cm TROCADE-loaded collagen surgical implant would typically not exceed 120 mg of TROCADE (or less than the established well tolerated single dose of 150 mg). Regardless of the size or type of collagen implant employed (surgical mesh, sling or patch), the total drug content should typically not exceed 150 mg of TROCADE. In one embodiment, 0.001-30% TROCADE by weight is loaded into PLGA microspheres, which are in turn loaded into the collagen implant, to produce sustained release of the drug over a period ranging from several days to several months. Any source of collagen (e.g., porcine, bovine, human, or recombinant; crosslinked or non-crosslinked) is suitable to be combined with the above to produce the desired end product. Pharmaceutically acceptable analogues and derivatives of TROCADE are also suitable for use in this embodiment either alone or in combination with other MMPIs.

4. MMPI-Loaded Dental Implants

Implantable collagen is often used in dental procedures to fill tissue defects and to promote healing and tissue regeneration. The embodiment described below details compositions of metalloproteinase inhibitor-loaded collagen products and methods for their use in the treatment of common periodontal conditions according to the present invention.

Briefly, periodontal disease is an inflammatory disease of the supporting structures of the teeth, including the ligaments, cementum, periosteum, alveolar bone and adjacent gingiva that anchor the teeth in place. The condition begins with bleeding of the gums, but can progress to loosening of the teeth, receding gums, abscesses in pockets between the gums and the teeth, and necrotizing ulcerative gingivitis. In advanced stages, procedures such as gingivectomy, gingivoplasty, and correction of the bony architecture of the teeth may be required for treatment of the condition. Traditional treatment involves open-flap debridement of the periodontal pocket with removal of diseased cementum, periodontal ligament and alveolar bone that have been destroyed by periodontal infection. Unfortunately, epithelial tissue can occasionally migrate into the surgically created defect impairing proper healing of the cementum, ligament and bone.

Collagen implants have been developed in an attempt to control the healing process and optimize tissue regeneration. Commonly used implants include, e.g., BIOMEND, available from Sulzer Medica, Inc. (Houston, Tex.), which is a collagen membrane composed of compressed Type I collagen matrix derived from bovine Achilles tendon. The collagen membrane (supplied as sheets, e.g., 15 mm×20 mm; 20 mm×30 mm; and 30 mm×40 mm) is cut to the appropriate size and shape, hydrated and placed as a barrier between the overlying gingival tissue and the debrided periodontal defect; the barrier can be sutured in place, but this is not always required. The membrane is placed snugly against the tooth root and draped over the surrounding alveolar bone (extending at least 3 mm beyond the defect margins) to effectively maintain the regenerative space. Primary closure with mucoperiosteal flaps over the collagen membrane is important as exposure of the membrane to the oral cavity can result in premature degradation. The barrier prevents faster growing epithelial tissue from entering the region and allows the slower growing periodontal ligament and bone cells to repopulate the area and effect appropriate healing. The collagen membrane is bioresorbable, is retained for 6 to 7 weeks, and is fully absorbed by host enzymes (e.g., collagenase) within 8 weeks.

However, limited durability of the collagen implant can become a clinical problem if it completely absorbs prior to the completion of healing—this is particularly relevant with large tissue defects. In an attempt to address this problem, manufacturers have attempted to produce a collagen implant with improved durability through increased collagen crosslinking (often through exposure of the collagen to aldehydes). Utilizing this process, products such as BIOMEND® EXTEND (Sulzer Medica, Inc.) can function as a barrier for longer periods of time, such that the collagen is not absorbed into the surrounding tissue for approximately 18 weeks. Another collagen dental implant product, OSSIX™ (Colbar R&D Ltd., Israel), uses a metabolite to crosslink collagen and prolong the structural integrity of the matrix for periods of up to 6 months. However, despite these efforts, there remains a need for the production of collagen dental implants with sustained structural integrity and slower degradation times. Utilizing an MMPI-loaded collagen dental implant according to the present invention can sustain the activity of the barrier, prolong structural integrity of the matrix, and allow more effective healing of the periodontal tissue defect. The implant will still ultimately degrade, but will last for longer than currently available biodegradable collagen implants regardless of the degree (or type) of collagen crosslinking present. This embodiment is also superior to permanent implants, such as e-PTFE membranes (e.g., GORE-TEX), which can require a second operative procedure to remove the implant.

In addition to the commercially available collagen-based products for the management of periodontal disease described above, other types of collagen-based implants may be combined with an MMPI and used in the practice of the present invention. Representative examples of such implants include those that are used in variety of dental procedures including: COLLATAPE® (Sulzer Medica, Inc.), which is a collagen-based implant used in the repair of minor oral wounds, closure of grafted sites and repair of Schneiderian Membranes; COLLACOTE® (Sulzer Medica, Inc.), a collagen-based wound dressing used for palatal donor sites and in mucosal flaps; and COLLAPLUG®0 (Sulzer Medica, Inc.), a solid collagen-based implant used in the repair of larger tissue defects such extraction sites or biopsy sites.

In the present invention, an MMPI may be added to the collagen-based dental implant in a sustained-release form to decrease the rate of degradation of the implant and prolong its activity in vivo beyond that seen with collagen alone (e.g. consistently greater than 10 weeks for certain applications (such as oral wounds, grafted sites, repair of Schneiderian membranes), beyond 20 weeks in other applications (such as mucosal flaps, periodontal disease without alveolar bone loss, periodontal disease with minor bone loss), and for 6 months to a year for other indications (such as periodontal disease with significant alveolar bone loss)).

Any MMPI described above could be utilized alone, or in combination, in the practice of this embodiment. Preferred MMPI's for use in dental implants include TIMP-1, tetracycline, doxycycline, minocycline, and other chemically-modified tetracyclines (CMTs), BATIMASTAT, MARIMASTAT, RO-1130830, CGS 27023A, BMS-275291, CMT-3, SOLIMASTAT, ILOMASTAT, CP-544439, PRINOMASTAT, PNU-1427690, SU-5402 and TROCADE, as well as analogues and derivatives of the aforementioned. The total dose delivered, the rate of dose release, and the duration of drug release from the matrix can be tailored to achieve variable degradation times of the collagen implant as required. The following compositions are ideally suited for use as dental implants:

a. MARIMASTAT-Loaded Collagen Dental Implants

A preferred MARIMASTAT-loaded dental collagen implant is 0.001 % -30% MARIMASTAT by weight (i.e., 1 μg-30 mg MARIMASTAT per 100 mg of collagen implant). A particularly preferred dosage is 0.01-15% MARIMASTAT by weight (i.e., 10 μg-15 mg per 100 mg of collagen implant). Alternatively, since the material is often packaged as a sheet (typical sizes are 15 mm×20 mm; 20 mm×30 mm; and 30 mm×40 mm) drug dosage can also be determined as a function of area. Preferred dosing of MARIMASTAT using this dosing regimen is 1 μg-37.5 μg/mm² of collagen implant. The total dosage delivered in periodontal treatment would typically not exceed 45 mg (or less than the established well tolerated single daily does of 50 mg). Regardless of the size or type of collagen implant employed (sheet, tape, plug or tissue filler) the total drug content should typically not exceed 50 mg of MARIMASTAT. In one embodiment, 0.001-30% MARIMASTAT by weight is loaded into PLGA microspheres, which are in turn loaded into the collagen implant, to produce sustained release of the drug over a period ranging from several days to several months. Any source of collagen (e.g., porcine, bovine, human, or recombinant; crosslinked or non-crosslinked) is suitable to be combined with the above to produce the desired end product. Pharmaceutically acceptable analogues and derivatives of MARIMASTAT are also suitable for use in this embodiment either alone or in combination with other MMPIs.

b. BATIMISTAT-Loaded Collagen Dental Implants

A preferred composition is 0.001-30% BATIMISTAT by weight (i.e., 1 ug-30 mg BATIMISTAT per 100 mg of collagen implant). A particularly preferred dosage is 0.01 to 30% by weight (10 μg-30 mg per 100 mg of collagen implant). Alternatively, since the material is often packaged as a sheet (typical sizes are 15 mm×20 mm; 20 mm×30 mm; and 30 mm×40 mm) drug dosage can also be determined as a function of area. Preferred dosing of BATIMISTAT using this dosing regimen is 1 μg-200 μg/mm² of collagen implant. Therefore, the total dosage delivered in a 30 mm×40 mm BATIMISTAT-loaded collagen dental implant would typically not exceed 240 mg of BATIMISTAT (or less than the established well tolerated single dose of 300 mg/m²). Regardless of the size or type of collagen implant employed (sheet, tape, plug or tissue filler) the total drug content should typically not exceed 300 mg of BATIMISTAT. In one embodiment, 0.001-30% BATIMISTAT by weight is loaded into PLGA microspheres, which are in turn loaded into the collagen implant, to produce sustained release of the drug over a period ranging from several days to several months. Any source of collagen (e.g., porcine, bovine, human, or recombinant; crosslinked or non-crosslinked) is suitable to be combined with the above to produce the desired end product. Pharmaceutically acceptable analogues and derivatives of BATIMISTAT are also suitable for use in this embodiment either alone or in combination with other MMPIs.

c. Doxycycline-Loaded Collapen Dental Implants.

A preferred composition is 0.001-30% doxycycline by weight (1 μg-30 mg doxycycline per 100 mg of collagen implant). A particularly preferred dosage is 0.01-30% doxcycline by weight (10 μg-30 mg doxycycline per 100 mg of collagen implant). Alternatively, since the material is often packaged as a sheet (typical sizes are 15 mm×20 mm; 20 mm×30 mm; and 30 mm×40 mm) drug dosage can also be determined as a function of area. Preferred dosing of doxycycline using this dosing regimen is 1 μg-83 μg/mm² of collagen implant. The total dosage delivered in a 30 mm×40 mm doxycycline-loaded collagen dental implant would typically not exceed 100 mg of doxycycline (or less than the established well tolerated single dose of 200 mg). Regardless of the size or type of collagen implant employed (sheet, tape, plug or tissue filler) the total drug content should typically not exceed 200 mg of doxycycline. In one embodiment, 0.001-30% doxycycline by weight is loaded into PLGA microspheres, which are in turn loaded into the collagen implant, to produce sustained release of the drug over a period ranging from several days to several months. Any source of collagen (e.g., porcine, bovine, human, or recombinant; crosslinked or non-crosslinked) is suitable to be combined with the above to produce the desired end product. Pharmaceutically acceptable analogues and derivatives of doxycycline are also suitable for use in this embodiment either alone or in combination with other MMPIs.

d. Tetracycline-Loaded Collagen Dental Implants

A preferred composition is 0.001-30% tetracycline by weight (1 μg-30 mg tetracycline per 100 mg of collagen implant). A particularly preferred dosage is 0.01-30% tetracycline by weight (10 μg-30 mg tetracycline per 100 mg of collagen implant). Alternatively, since the material is often packaged as a sheet (typical sizes are 15 mm×20 mm; 20 mm×30 mm; and 30 mm×40 mm) drug dosage can also be determined as a function of area. Preferred dosing of tetracycline using this dosing regimen is 1 μg-625 μg/mm² of collagen implant. The total dosage delivered in a 30 mm×40 mm tetracycline-loaded collagen dental implant would typically not exceed 750 mg of tetracycline (or less than the established well tolerated single dose of 1000 mg). Regardless of the size or type of collagen implant employed (sheet, tape, plug or tissue filler) the total drug content should typically not exceed 1000 mg of tetracycline. In one embodiment, 0.001-30% tetracycline by weight is loaded into PLGA microspheres, which are in turn loaded into the collagen implant, to produce sustained release of the drug over a period ranging from several days to several months. Any source of collagen (e.g., porcine, bovine, human, or recombinant; crosslinked or non-crosslinked) is suitable to be combined with the above to produce the desired end product. Pharmaceutically acceptable analogues and derivatives of tetracycline, including chemically-modified tetracylines (CMTs), are also suitable for use in this embodiment either alone or in combination with other MMPIs.

e. Minocycline-Loaded Collagen Dental Implants

A preferred composition is 0.001-30% minocycline by weight (1 μg-30 mg minocycline per 100 mg of collagen implant). A particularly preferred dosage is 0.01-6% minocycline by weight (10 μg-6 mg minocycline per 100 mg of collagen implant). Alternatively, since the material is often packaged as a sheet (typical sizes are 15 mm×20 mm; 20 mm×30 mm; and 30 mm×40 mm) drug dosage can also be determined as a function of area. Preferred dosing of minocycline using this dosing regimen is 1 μg-150 μg/mm² of collagen implant. The total dosage delivered in a 30 mm×40 mm minocycline-loaded collagen dental implant would typically not exceed 180 mg of minocycline (or less than the established tolerated single dose of 200 mg). Regardless of the size or type of collagen implant employed (sheet, tape, plug or tissue filler) the total drug content should typically not exceed 200 mg of minocycline. In one embodiment, 0.01-30% minocycline is loaded into PLGA microspheres, which are in turn loaded into the collagen implant, to produce sustained release of the agent over a period ranging from several days to several months. Any source of collagen (e.g., porcine, bovine, human, or recombinant; crosslinked or non-crosslinked) is suitable to be combined with the above to produce the desired end product. Pharmaceutically acceptable analogues and derivatives of minocycline are also suitable for use in this embodiment either alone or in combination with other MMPIs.

f. TROCADE-Loaded Collagen Dental Implants

A preferred composition is 0.001-30% TROCADE by weight (1 μg-30 mg TROCADE per 100 mg of collagen implant). A particularly preferred dosage is 0.01 to 5% TROCADE by weight (10 μg-5 mg TROCADE per 100 mg of collagen implant). Alternatively, since the material is often packaged as a sheet (typical sizes are 15 mm×20 mm; 20 mm×30 mm; and 30 mm×40 mm) drug dosage can also be determined as a function of area. Preferred dosing of TROCADE using this dosing regimen is 1 μg-100 μg/mm² of collagen implant. The total dosage delivered in a 30 mm×40 mm TROCADE-loaded collagen dental implant would typically not exceed 120 mg of TROCADE (or less than the established well tolerated single dose of 150 mg). Regardless of the size or type of collagen implant employed (sheet, tape, plug or tissue filler) the total drug content should typically not exceed 150 mg of TROCADE. In one embodiment, 0.001-30% TROCADE by weight is loaded into PLGA microspheres, which are in turn loaded into the collagen implant, to produce sustained release of the drug over a period ranging from several days to several months. Any source of collagen (e.g., porcine, bovine, human, or recombinant; crosslinked or non-crosslinked) is suitable to be combined with the above to produce the desired end product. Pharmaceutically acceptable analogues and derivatives of TROCADE are also suitable for use in this embodiment either alone or in combination with other MMPIs.

5. MMPI-Loaded Collagen Skin Grafts

Several collagen-based products have been developed for use as artificial skin grafts. ORCEL™ Bilayered Cellular Matrix (Ortec International, Inc., New York, N.Y.) is composed of purified bovine Type I collagen mixed with two types of living human skin cells. ORCEL™ is a wound dressing applied to the wound surface to promote healing before gradually being absorbed. A related product, Composite Cultured Skin (Ortec International, Inc.), is a wound dressing composed of purified bovine Type I collagen mixed with human skin cells taken from healthy donors for use in the management of recessive dystrophic epidermolysis bullosa (RDEB). APLIGRAF® (Organogenesis Inc., Canton, Mass.) is a living, bilayered skin substitute manufactured using neonatal foreskin keratinocytes and fibroblasts with bovine Type I collagen. It is indicated for the treatment of partial and/or full-thickness skin ulcers such as venous leg ulcers and diabetic foot ulcers. Representative examples of skin grafts and methods for preparing artificial skin are described in U.S. Pat. Nos. 5,166,187, 5,263,983, 5,326,356, 5,350,583, 5,800,811, and 5,945,101.

According to the present invention, differential loading of an MMPI into a collagen-based skin graft may be used for accurately controlling the dissolution rate of the graft. Thus, the present invention provides a collagen-based skin graft in combination with an MMPI. Although any of the previously described metalloproteinase inhibitors could be suitable for incorporation into a collagen-based skin graft, the following are particularly preferred: TIMP-1, tetracycline, chemically-modified tetraclines (CMTs), doxycycline, minocycline, BATIMASTAT, MARIMASTAT, RO-1130830, CGS 27023A, BMS-275291, CMT-3, SOLIMASTAT, ILOMASTAT, CP-544439, PRINOMASTAT, PNU-1427690, SU-5402 and TROCADE, as well as analogues or derivatives of the aforementioned. By varying the amount of the MMPI loaded into the collagen-based skin graft from 0.001-30% by weight (1 μg-30 mg per 100 mg of collagen), dissolution can be varied from 12 hours to 72 hours and beyond. Any source of collagen (e.g., porcine, bovine, human, or recombinant; crosslinked or non-crosslinked) is suitable for production of the above product.

6. MMPI-Loaded Collagen Corneal Shields

Corneal shields are used post-operatively, usually following cataract surgery, to function as a splint to facilitate healing by immobilizing and protecting scleral and conjunctival tissue. Corneal shields provide continuous lubrication to compromised tissue while providing a protective barrier and increasing patient comfort. Varieties of collagen-based corneal shields are available for this clinical use and differ primarily by their duration of activity. The SURGILENS™ shield (Bausch & Lomb, Inc., Rochester, N.Y.) is a rapidly dissolving lens that is completely resorbed in 12 hours. Oasis Medical Inc. (Glendora, Calif.) makes several different collagen corneal shields including: the SOFT SHIELD® QS; the SOFT SHIELD®, 12-hour (12 hour dissolution time); the SOFT SHIELD, 24-hour (24 hour dissolution time); and the SOFT SHIELD®, 72-hour (72 hour dissolution time). Alcon Laboratories, Inc. (Fort Worth, Tex.) also manufactures a line of collagen corneal shields, known as PROSHIELD®, that are available in a wide range of dissolution rates. Representative examples of corneal shields are described in U.S. Pat. Nos. 6,106,554 5,128,134, 5,094,856, 5,094,855, 5,093,125, and 4,913,904.

According to the present invention, differential loading of an MMPI into a collagen corneal shield may be used for accurately controlling the dissolution rate of the shield. Thus, in one embodiment, the present invention provides a collagen-containing corneal shield in combination with an MMPI. Although any of the previously described metalloproteinase inhibitors could be suitable for incorporation into a collagen corneal shield, the following are particularly preferred: TIMP-1, tetracycline, chemically-modifed tetracylines (CMTs), doxycycline, minocycline, BATIMASTAT, MARIMASTAT, RO-1130830, CGS 27023A, BMS-275291, CMT-3, SOLIMASTAT, ILOMASTAT, CP-544439, PRINOMASTAT, PNU-1427690, SU-5402 and TROCADE, as well as analogues and derivatives of the aforementioned. By varying the amount of the MMPI loaded into the collagen corneal shield from 0.001-30% by weight (1 μg-30 mg per 100 mg of collagen), dissolution can be varied from 12 hours to 72 hours and beyond. Any source of collagen (e.g., porcine, bovine, human, or recombinant; crosslinked or non-crosslinked) is suitable for production of the above product.

7. MMPI-Loaded Collagen Glaucoma Drainage Devices

Collagen-based glaucoma drainage devices are used in the surgical management of open-angle glaucoma. Glaucoma is a common eye condition in which pressure within the eyeball (intraocular pressure-IOP) increases to the point where retinal tissues can be damaged (occasionally to the point of causing blindness). When medications are ineffective, surgery may be required to facilitate drainage of the aqueous humor and reduce intraocular pressure. Non-penetrating deep sclerectomy is performed to provide an alternative route for aqueous fluid drainage and to reduce pressure. Cylindrical tubes, such as the AQUAFLOW Collagen Glaucoma Drainage Device (STAAR Surgical Company, Monrovia, Calif.), are used to maintain the subscleral drainage channel. The AQUAFLOW is 4.0 mm long by 0.5 mm wide (when dry) and is composed entirely of lyophilized, cross-linked porcine collagen. After placement, the device absorbs fluid and swells to fill the surgically created space to allow ongoing drainage of the aqueous humor. Over time, the device begins to slowly dissolve until it is completely resorbed within 6-9 months. Representative examples of glaucoma drainage devices are described in U.S. Pat. Nos. 4,722,724, 5,178,604 and 5,893,837.

Loading an MMPI into a collagen glaucoma drainage device may be used for slowing the dissolution rate of the implant and prolonging its effectiveness beyond 6-9 months. Thus, in one embodiment, the present invention provides a collagen-containing glaucoma drainage device in combination with an MMPI. Although any of the previously described metalloproteinase inhibitors could be suitable for incorporation into a collagen glaucoma drainage device, the following are particularly preferred: TIMP-1, tetracycline, chemically-modified tetracycline, doxycycline, minocycline, BATIMISTAT, MARIMASTAT, RO-1130830, CGS 27023A, BMS-275291, CMT-3, SOLIMASTAT, ILOMASTAT, CP-544439, PRINOMASTAT, PNU-1427690, SU-5402 and TROCADE, as well as analogues and derivatives of the aforementioned. By varying the amount of the MMPI loaded into the collagen glaucoma drainage device from 1-30% by weight, the effective lifespan of the device can be increased beyond 9 months. In one embodiment, 1-30% of TIMP-1, tetracycline, doxycycline, minocycline, BATIMASTAT, MARIMASTAT, RO-1130830, CGS 27023A, BMS-275291, CMT-3, SOLIMASTAT, ILOMASTAT, CP-544439, PRINOMASTAT, PNU-1427690, SU-5402 and/or TROCADE (by weight) is/are loaded into PLGA microspheres, which are in turn loaded into the collagen cylinder, to produce sustained release of the drug over a period of several months.

8. MMPI-Loaded Collagen Bulking Agents for GERD

Collagen-based injectables are used for the management of gastroesophageal reflux disease (GERD). GERD occurs when the lower esophageal sphincter (the muscle between the stomach and the esophagus) is unable to prevent the contents of the stomach from refluxing back into the esophagus. Gastric acid and enzymes are quite corrosive to the epithelial lining of the esophagus and can cause erosions, ulceration, scarring and narrowing of the esophagus. Repetitive reflux into the esophagus can result in irreversible injury and also predisposes the patient to the development of esophageal cancer. Injection of a collagen-bulking agent into the vicinity of the lower esophageal sphincter (LES) can restore the structure of the tissue and reduce backflow into the esophagus. The collagen-bulking agent is typically administered through direct injection under endoscopic vision. As occurs with virtually all collagen-based procedures, the principle problem is degradation of the implant, which limits the longevity of the treatment. A repeat intervention, with either reinjection of collagen or open surgical reinforcement of the sphincter, is required when the collagen loses its structural integrity and can no longer maintain the LES.

In the present invention, an MMPI is added to the collagen-based injection in a sustained-release form to decrease the rate of degradation of the LES implant and prolong its activity in vivo beyond that seen with collagen alone (e.g. consistently longer than 1 year in >75% of patients and longer than 3 years in >35% of patients). Any MMPI described previously could be utilized alone, or in combination, for the practice of this embodiment. Preferred MMPI's for use in injectable collagen implants for GERD include TIMP-1, tetracycline, doxycycline, minocycline, and other chemically-modified tetracyclines (CMTs), BATIMASTAT, MARIMASTAT, RO-1130830, CGS 27023A, BMS-275291, CMT-3, SOLIMASTAT, ILOMASTAT, CP-544439, PRINOMASTAT, PNU-1427690, SU-5402 and TROCADE as well as analogues and derivatives of the aforementioned. The total dose delivered, the rate of dose release, and the duration of drug release from the matrix can be tailored to significantly prolong the activity of the collagen implant as required. The following compositions are ideally suited for use in this indication:

a. MARIMASTAT-Loaded Collagen Bulking Agents for GERD

A preferred MARIMASTAT-loaded injectable collagen implant is 0.001%-30% MARIMASTAT by weight (i.e., 1 μg-30 mg MARIMISTAT per 100 mg of collagen injected). A particularly preferred dosage is 0.01-15% MARIMASTAT by weight (i.e., 10 μg-15 mg per 100 mg of collagen implanted). Regardless of the size or type of collagen implant injected, the total drug content should typically not exceed 50 mg of MARIMASTAT. In one embodiment, 0.001-30% MARIMASTAT by weight is loaded into PLGA microspheres, which are in turn loaded into the collagen implant, to produce sustained release of the drug over a period ranging from several days to several months. Any source of collagen (e.g., porcine, bovine, human, or recombinant; crosslinked or non-crosslinked) is suitable to be combined with the above to produce the desired end product. Pharmaceutically acceptable analogues and derivatives of MARIMASTAT are also suitable for use in this embodiment either alone or in combination with other MMPIs.

b. BATIMASTAT-Loaded Collagen Bulking Agents for GERD

A preferred composition is 0.001-30% BATIMASTAT by weight (i.e., 1 μg-30 mg BATIMASTAT per 100 mg of collagen injected). A particularly preferred dosage is 0.01 to 30% by weight ( l0ig-30 mg per 100 mg of collagen implanted). Regardless of the size or type of collagen implant employed, the total drug content should typically not exceed 300 mg of BATIMASTAT. In one embodiment, 0.01-30% BATIMASTAT by weight is loaded into PLGA microspheres, which are in turn loaded into the collagen implant, to produce sustained release of the drug over a period ranging from several days to several months. Any source of collagen (e.g., porcine, bovine, human, or recombinant; crosslinked or non-crosslinked) is suitable to be combined with the above to produce the desired end product. Pharmaceutically acceptable analogues and derivatives of BATIMASTAT are also suitable for use in this embodiment either alone or in combination with other MMPIs.

c. Doxycycline-Loaded Collagen Bulking Agents for GERD

A preferred composition is 0.001-30% doxycycline by weight (1 μg-30 mg doxycycline per 100 mg of collagen injected). A particularly preferred dosage is 0.01-30% doxcycline by weight (10 μg-30 mg doxycycline per 100 mg of collagen implanted). Regardless of the size or type of collagen implant employed, the total drug content should typically not exceed 200 mg of doxycycline. In one embodiment, 0.001-30% doxycycline by weight is loaded into PLGA microspheres, which are in turn loaded into the collagen implant, to produce sustained release of the drug over a period ranging from several days to several months. Any source of collagen (e.g., porcine, bovine, human, or recombinant; crosslinked or non-crosslinked) is suitable to be combined with the above to produce the desired end product. Pharmaceutically acceptable analogues and derivatives of doxycycline are also suitable for use in this embodiment either alone or in combination with other MMPIs.

d. Tetracycline-Loaded Collagen Bulking Agents for GERD

A preferred composition is 0.001-30% tetracycline by weight (1 μg-30 mg tetracycline per 100 mg of collagen injected). A particularly preferred dosage is 0.01-30% tetracycline by weight (10 μg-30 mg tetracycline per 100 mg of collagen implanted). Regardless of the size or type of collagen implant employed, the total drug content should typically not exceed 1000 mg of tetracycline. In one embodiment, 0.001-30% tetracycline by weight is loaded into PLGA microspheres, which are in turn loaded into the collagen implant, to produce sustained release of the drug over a period ranging from several days to several months. Any source of collagen (e.g., porcine, bovine, human, or recombinant; crosslinked or non-crosslinked) is suitable to be combined with the above to produce the desired end product. Pharmaceutically acceptable analogues and derivatives of tetracycline, including chemically-modified tetracylines (CMTs), are also suitable for use in this embodiment either alone or in combination with other MMPIs.

e. Minocycline-Loaded Collagen Bulking Agents for GERD

A preferred composition is 0.001-30% minocycline by weight (1 μg-30 mg minocycline per 100 mg of collagen injected). A particularly preferred dosage is 0.01-6% minocycline by weight (10 μg-6 mg minocycline per 100 mg of collagen implanted). Regardless of the size or type of collagen implant employed, the total drug content should typically not exceed 200 mg of minocycline. In one embodiment, 0.001-30% minocycline is loaded into PLGA microspheres, which are in turn loaded into the collagen implant, to produce sustained release of the agent over a period ranging from several days to several months. Any source of collagen (e.g., porcine, bovine, human, or recombinant; crosslinked or noncrosslinked) is suitable to be combined with the above to produce the desired end product. Pharmaceutically acceptable analogues and derivatives of minocycline are also suitable for use in this embodiment either alone or in combination with other MMPIs.

f. TROCADE-Loaded Collagen Bulking Agents for GERD

A preferred composition is 0.001-30% TROCADE by weight (1 μg-30 mg TROCADE per 100 mg of collagen injected). A particularly preferred dosage is 0.01 to 5% TROCADE by weight (10 μg-5 mg TROCADE per 100 mg of collagen implanted). Regardless of the size or type of collagen implant employed, the total drug content should typically not exceed 150 mg of TROCADE. In one embodiment, 0.001-30% TROCADE by weight is loaded into PLGA microspheres, which are in turn loaded into the collagen implant, to produce sustained release of the drug over a period ranging from several days to several months. Any source of collagen (e.g., porcine, bovine, human, or recombinant; crosslinked or noncrosslinked) is suitable to be combined with the above to produce the desired end product. Pharmaceutically acceptable analogues and derivatives of TROCADE are also suitable for use in this embodiment either alone or in combination with other MMPIs.

9. MMPI-Loaded Collagen Bulking Agents for Fecal Incontinence

Collagen-based injectables may also be used in the local management of fecal incontinence. Fecal incontinence is a common and socially disabling condition that affects up to 11% of North American adults. Incontinence to flatus or feces can be caused by a variety if factors, but is more common in women where the anal sphincter can be damaged during child birth (especially those who have suffered a third degree vaginal tear, required forceps, had large babies, and/or experienced long labor as part of a vaginal delivery). Although the etiology of fecal incontinence is often multifactorial, causes include sphincter injury (obstetric, surgical, accidental), anorectal disease (hemorrhoids, rectal prolapse, inflammatory bowel disease, fistulas, tumors, colon resection, fecal impaction, diarrhea), congenital (spina bifida, meningocele, Hirshsprung's disease), idiopathic, or behavioral (resistance to defecation, dementia, mental retardation). Passive fecal incontinence (i.e., occurring without the patient's awareness) is primarily due to dysfunction of the internal anal sphincter, while urge fecal incontinence (the inability to voluntarily suppress defecation) is usually due to external anal sphincter dysfunction.

Corrective measures are initially conservative or directed towards eliminating the underlying cause (if readily evident). In a significant number of patients, no defined cause can be identified and surgical repair of the internal or external anal sphincter is often attempted. Unfortunately, over 50% of these patients will not achieve a long-term successful outcome and will require another form of treatment. Those who have failed surgery, patients who do not wish to have surgery, and patients who cannot be operated on for medical reasons are all candidates for injectable sphincter augmentation. In this procedure a bulking agent, typically collagen, is injected into the region around the internal or external sphincter to increase sphincter pressure and reduce fecal incontinence.

Although peri-anal-sphincter collagen injections have been used with a great deal of success in the management of fecal incontinence, the majority of cases require more than one treatment due to the limited durability of the collagen implant. Utilizing an MMPI-loaded collagen injection according to the present invention can sustain the activity of the implant and reduce the need for, and frequency of, subsequent peri-anal-sphincter injections.

Several commercially available collagen-based bulking agents are available for the management of fecal incontinence. CONTIGEN® (purified bovine dermal glutaraldehyde crosslinked collagen dispersed in phosphate buffered physiologic saline at 35 mg/ml available through CR Bard) is a widely used bulking agent. Other collagen based injectable products, including those derived from non-bovine, human, or recombinant sources can also be utilized in this embodiment. With CONTIGEN®, the crosslinked collagen begins to degrade in approximately 12 weeks and degrades completely within 10 to 19 months. Although the percentage of patients showing improvement in their fecal incontinence after collagen injection is initially quite high, gradual resorption of the collagen results in the need to repeat the procedure in the majority of patients. In the present invention, an MMPI is added to the collagen-based injectable in a sustained-release form to decrease the rate of degradation of the implant and prolong its activity in vivo beyond that seen with collagen alone (i.e., consistently greater than 6 months in the majority of patients and beyond 1 year in a significant percentage of others).

Peri-anal-sphincter injection of an MMPI-loaded collagen is performed in the following manner. Prior to administration of the material, the patient should have completed two skin tests (conducted 2 weeks apart) to test for an allergic response. If these tests are negative, the MMPI-loaded collagen injection can be administered to the patient. A refrigerated, single use, pre-loaded syringe with a fine gauge needle containing 2.5 mL of the implant material is used. The patient is placed in the lithotomy position, 10 mL of 2% lidocaine is inserted into the perineal skin or the rectal mucosa depending upon the region of injection selected. The needle is inserted through the skin or the rectal mucosa into the submucosal plane surrounding the anal sphincter. When needle reaches the appropriate position, the MMPI-loaded collagen is injected slowly into the site (typically in 3 injections placed circumferentially, trans-sphincterally, entering away from the anal margin and injecting at, or just above, the dentate line) until symmetry is achieved around the anal canal. Methylene blue, or other nontoxic coloring agents, can be added to the implant to assist with visualization of the injection.

Although potentially any MMPI-loaded collagen injection could be suitable for the treatment of fecal incontinence, MMPI's such as TIMP-1, tetracycline, doxycycline, minocycline, BATIMISTAT, MARIMISTAT, Ro-1130830, CGS 27023A, BMS-275291, CMT-3, Solimastat, Ilomastat, CP-544439, Prinomastat, PNU-1427690, SU-5402, and TROCADE are particularly preferred. The following compositions are ideally suited for use as anal sphincter bulking agents:

a. MARIMISTAT-Loaded Collagen Anal Sphincter Bulking Agents

A preferred composition is 0.001% -30% MARIMISTAT per cc (i.e., 1 μg-30 mg MARIMISTAT by weight) of collagen/saline suspension. A particularly preferred dosage is 0.01-15% MARIMISTAT (i.e., 10 μg to 15 mg) per mL of collagen/saline suspension. Therefore, the total dosage delivered in a 2.5 mL treatment would typically not exceed 45 mg (or less than the established well tolerated single daily does of 50 mg). In one embodiment, 0.001-30% MARIMISTAT is loaded into PLGA microspheres or other polymer-based microspheres which are in turn loaded into the collagen, in order to produce sustained release of the material over a period ranging from several days to several months. Any source of injectable collagen (e.g., bovine, human, or recombinant; crosslinked or noncrosslinked) would be suitable to be combined with the above to produce the desired end product. Pharmaceutically acceptable analogues and derivatives of MARIMISTAT are also suitable for use in this embodiment either alone or in combination with other MMPIs.

b. BATIMISTAT-Loaded Collagen Anal Sphincter Bulking Agents

A preferred composition is 0.001 to 30% BATIMISTAT (i.e., 1 μg to 30 mg BATIMISTAT by weight) per mL of injectable collagen/saline suspension. A particularly preferred dosage is 0.01 to 30% (10 μg to 30 mg by weight) per mL of collagen/saline suspension. Therefore, the total dosage delivered in a 2.5 mL treatment would typically not exceed 75 mg of BATIMISTAT (or less than the established well tolerated single dose of 300 mg/M2). In one embodiment, 0.001 to 30% BATIMISTAR is loaded into PLGA microspheres or other polymer-based microspheres which are in turn loaded into the collagen, in order to produce sustained release of the agent over a period ranging from several days to several months. Any source of injectable collagen (e.g., bovine, human, or recombinant; crosslinked or noncrosslinked) would be suitable to be combined with the above to produce the desired end product. Pharmaceutically acceptable analogues and derivatives of BATIMISTAT are also suitable for use in this embodiment either alone or in combination with other MMPIs.

c. Doxycycline-Loaded Collagen Anal Sphincter Bulking Agents

A preferred composition is 0.001-30% doxycycline (1 μg to 30 mg doxycycline by weight) per mL of injectable collagen/saline suspension. A particularly preferred dosage is 0.01 to 30% doxcycline (10 μg to 30 mg doxycycline by weight) per mL of collagen/saline suspension. Therefore the total dosage administered in a 2.5 mL treatment would typically not exceed 75 mg (or less than the well tolerated daily dosage of 100 mg). In one embodiment 0.001% to 30% doxycycline is loaded into PLGA microspheres or other polymer-based microspheres which are in turn loaded into the collagen, in order to produce sustained release of the agent over a period ranging from several days to several months. Any source of injectable collagen (e.g., bovine, human, or recombinant; crosslinked or noncrosslinked) would be suitable to be combined with the above to produce the desired end product. Pharmaceutically acceptable analogues and derivatives of DOXYCYCLINE are also suitable for use in this embodiment either alone or in combination with other MMPIs.

d. Tetracycline-Loaded Collagen Anal Sphincter Bulking Agents

A preferred composition is 0.001-30% tetracycline (1 μg to 30 mg tetracycline by weight) per mL of injectable collagen/saline suspension. A particularly preferred dosage is 0.01 to 30% tetracycline (10 μg to 30 mg tetracycline by weight) per mL of collagen/saline suspension. Therefore the total dosage administered in a 2.5 mL treatment would typically not exceed 75 mg (or less than the well tolerated daily dosage of 1 g). In one embodiment 0.001% to 30% tetracycline is loaded into PLGA microspheres or other polymer-based microspheres which are in turn loaded into the collagen, in order to produce sustained release of the agent over a period ranging from several days to several months. Any source of injectable collagen (e.g., bovine, human, or recombinant; crosslinked or noncrosslinked) would be suitable to be combined with the above to produce the desired end product. Pharmaceutically acceptable analogues and derivatives of TETRACYCLINE are also suitable for use in this embodiment either alone or in combination with other MMPIs.

e. Minocycline-Loaded Collagen Anal Sphincter Bulking Agents

A preferred composition is 0.001-30% minocycline (1 μg to 30 mg tetracycline by weight) per mL of injectable collagen/saline suspension. A particularly preferred dosage is 0.01 to 6% minocycline (10 μg to 6 mg minocycline by weight) per cc of collagen/saline suspension. Therefore the total dosage administered in a 30 cc treatment would typically not exceed 180 mg or less than the well tolerated daily dosage of 200 mg). In one embodiment 0.001% to 30% minocycline is loaded into PLGA microspheres or other polymer-based microspheres which are in turn loaded into the collagen, in order to produce sustained release of the agent over a period ranging from several days to several months. Any source of injectable collagen (e.g., bovine, human, or recombinant; crosslinked or noncrosslinked) would be suitable to be combined with the above to produce the desired end product. Pharmaceutically acceptable analogues and derivatives of MINOCYCLINE are also suitable for use in this embodiment either alone or in combination with other MMPIs.

f. TROCADE-Loaded Collagen Anal Sphincter Bulking Agents

A preferred composition is 0.001-30% TROCADE (1 μg to 30 mg TROCADE by weight) per mL of injectable collagen/saline suspension. A particularly preferred dosage is 0.01 to 5% TROCADE (10 μg to 5 mg TROCADE by weight) per ml of collagen/saline suspension. Therefore the total dosage administered in a 2.5 mL treatment would typically not exceed 75 mg. In one embodiment 0.001% to 30% TROCADE is loaded into PLGA microspheres or other polymer-based microspheres which are in turn loaded into the collagen, in order to produce sustained release of the agent over a period ranging from several days to several months. Any source of injectable collagen (e.g., bovine, human, or recombinant; crosslinked or noncrosslinked) would be suitable to be combined with the above to produce the desired end product. Pharmaceutically acceptable analogues and derivatives of TROCADE are also suitable for use in this embodiment either alone or in combination with other MMPIs.

It should be readily evident to one of skill in the art that any of the previously described MMPI agents, or derivatives and analogues thereof, can be utilized to create variations of the above compositions without deviating from the spirit and scope of the invention. It should also be apparent that the MMPI can be utilized in a collagen implant with or without polymer carrier and that altering the carrier does not deviate from the scope of this invention.

### EXAMPLES

### Example 1

### Preparation of Collagen

Collagen Source

Skin is removed from freshly sacrificed rabbits. The removed skin is shaved, defatted by sharp dissection and cut into two cm² squares. The skin squares are freeze-dried at ambient temperature for 24 hours and then ground, with the aid of solid CO₂, in a mill to produce a powder.

Solubilization

A suspension of the powdered skin in prepared by adding the powdered material to a 0.5 M acetic acid solution such that the skin concentration is 5 g dry wt skin/l. The suspension is cooled to 10° C. A freshly prepared pepsin solution (0.5 g in 10 ml 0.01 N HCl) is added to the skin suspension and the mixture was incubated for 5 days at 10° C. with occasional stirring.

Pepsin Removal

Following the enzymatic treatment, the remaining pepsin in the mixture was denatured by adding 5 ml Tris base and adjusting the pH to 7.0 with 3 N NaOH at 4° C. 30 g NaCl is stirred into the mixture to keep the collagen in solution. After 4 hours, the mixture is centrifuged at 30,000 g for 30 minutes to remove the precipitated pepsin.

Purification

The enzymatically treated collagen is precipitated from the supernatant liquid by adding an additional 140 g NaCl. The solution is stirred and allowed to stand for 4 hours at 4° C. The precipitated collagen is centrifuged out at 30,000 g for 30 minutes. The resulting collagen pellet is resuspended in 200 ml deionized water. 0.5 N acetic acid is added to bring the final volume to one liter. The collagen is precipitated from this solution by adding 50 g NaCl, allowing the solution to stand for 5 hours at 4° C. and centrifuging at 30,000 g for 30 minutes.

Sterilization

The collagen pellet is resuspended in 200 ml distilled water, transferred into sterilized dialysis tubing and dialysed for 72 hours against 50 volumes 1 N acetic acid. The collagen was then dialysed for 24 hours against 50 volumes 0.001 N acetic acid with the solution being changed 3 times during this period. The dialysed solution is then concentrated by placing the dialysis tube on sterile absorbant towels in a laminar-flow bacteriologic barrier until the concentration reached 12-15 mg collagen/ml solution. The concentrated solution is then dialysed against 50 volumes 0.001 N acetic acid for 24 hours. The collagen solution is then stored in sterile vials at 4° C.

Addition of Polymerization Promoter to Concentrate

Immediately prior to use a buffered salt solution (NaCl 2.5 mM/l, NaHPO₄ 0.1 mM/l, pH 7.4) is added at 4° C. to the collagen solution in a volume: volume ratio of 10:1 (collagen:buffer), and the buffered concentrate is transferred to a chilled (4° C.) syringe. For specific applications (e.g., cosmetic tissue augmentation), the buffered salt solution can also contain 0.3-1% (w/v) of a local anesthetic (e.g., Lidocaine).

### Example 2

### Prepartaion of TIMP-1-Loaded Microspheres Using a W/O/W Method

One hundred milligrams of 50/50 PLGA copolymer (IV=0.15) is added to 12 mL of dichloromethane. To this, add 800 μL of phosphate buffered saline (PBS) solution or TIMP-1 (concentration typically from 1 to 10 mg/mL) in PBS. This mixture is then homogenized (20 seconds at 6,000 rpm). Once formed this mixture is dispersed into 100 mL of a 1.0% aqueous solution of poly vinyl alcohol (PVA) and is immediately homogenized (40 seconds at 8,000 rpm) to form a water in oil in water double emulsion. Polydisperse microparticles (with the majority less than 10 microns in size) are formed under these conditions. The solvent is then slowly removed via evaporation and the microspheres collected by centrifugation. The particles are washed (5 times) with deionized water and then frozen in a dry ice/acetone bath and lyophilized overnight to yield a white freely flowing powder of microspheres.

Microspheres with a longer degradation profile are prepared using 85/15 PLGA (IV=0.68) using the method desribed above.

The method described above is also used to prepare microspheres containing TIMP-2, TIMP-3 and TIMP-4.

### Example 3

### Prepartaion of Tetracycline-Loaded Microspheres Using a W/O/W Method

Tetracycline-loaded microspheres are prepared in a similar manner to that described in the example above except that tetracycline hydrochloride is used.

### Example 4

### Preparation of Doxycycline-Loaded Microspheres Using a W/O/W Method

Doxycycline-loaded microspheres are prepared in a similar manner to that described in the example above except that doxycycline hydrochloride is used.

### Example 5

### Preparation of Minocycline-Loaded Microspheres Using a W/O/W Method

Minocycline-loaded microspheres are prepared in a similar manner to that described in the example above except that minocycline hydrochloride is used.

### Example 6

### Preparation of Batimistat-Loaded Microspheres Using an Oil-In-Water Method

PVA Solution Preparation

In a 1000 ml beaker, 1000 ml of distilled water and 100 g of PVA (Aldrich 13-23K, 98% hydrolyzed) are added. A two-inch stirrer bar is placed into the beaker. The suspension is heated up to 75-80° C. while stirring. Once the PVA is dissolved completely (forms a clear solution), the PVA solution (w/v) is cooled to room temperature and filtered through a syringe in-line filter.

PLGA Solution Preparation with BATIMASTAT

100 mg BATIMASTAT and 900 mg PLGA (50/50, IV=0.15) are weighed and transferred into the 20 ml scintillation vial. 10 mL of HPLC grade dichloromethane (DCM) is added to the vial to dissolve the PLGA and BATIMASTAT. The sample was place on an orbital shaker (setting 4) until the polymer and the BATIMASTAT were dissolved.

Preparation of the Microspheres with Diameter Less than 25 μm

100 ml of 10% PVA solution is transferred into a 400 ml beaker. The beaker is secured to the stand using double-sided adhesive tape. A 3-blade stirring rod blade is placed into the beaker and adjusted to a height of approx. 0.5 cm above the beaker bottom. The stirrer motor (DYNA-MIX from Fisher Scientific) is turned on to 2.5 at first. The 10 ml PLGA/BATIMASTAT solution is poured into the PVA solution during agitation. The stirring speed is the gradually increased to a setting of 5. The stirring is continued for 2.5 to 3.0 hours. The obtained microspheres were filtered through a 2 metal sieves (53 μm (top) and 25 μm (bottom)) into a 100 ml beaker in order to remove any large sized material. The microspheres are washed with distilled water while filtering. The microspheres that are collected in the filtrate were centrifuged (1000 rpm, 10 min.) to sediment the microspheres. The supernatant is removed using a pasteur pipette and the pellet is re-suspended with 100 ml distilled water. This process is repeated 2 additional times.

The washed microspheres are transferred into a glass container. The transfer is completed by rinsing the beaker with a small amount of distilled water (20-30 ml). The container is sealed with Parafilm and placed into a −20° C. freezer over night. The frozen microsphere solution is then freeze-dried using a freeze-drier for about 3 days. The dried microspheres are transferred into 20ml scintillation vial and were stored at −20° C. The microspheres are then terminally sterilized by irradiation with at least 2.5 Mrad Cobalt-60 (Co-60) x-rays.

### Example 7

### Preparation of Marmistat-Loaded Microspheres Using an Oil-In-Water Method

MARIMASTAT-loaded microspheres are prepared in a similar manner to that described in the example above, except that MARIMASTAT is used instead of BATIMASTAT.

### Example 8

### Preparation of Trocade-Loaded Microspheres Using an Oil-In-Water Method

TROCADE-loaded microspheres are prepared in a similar manner to that described in the example above, except that TROCADE is used instead of Batimistat.

### Example 9

### Manufacture Collagen Solution Containing Micellar Batimistat

Preparation of the Polymer

Polymer is synthesized using DL-lactide and methoxy poly(ethylene glycol) [MePEG 2000] in presence of 0.5% w/w stannous octoate through a bulk ring opening polymerization.

Reaction glassware is washed and rinsed with Sterile Water for Irrigation USP, dried at 37° C., followed by depyrogenation at 250° C. for at least I hour. MePEG 2000 and DL-lactide are weighed (240 g and 160 g, respectively) and transferred to a round bottom flask using a stainless steel funnel. A 2-inch Teflon® coated magnetic stir bar is added to the flask. A glass stopper is used to seal the flask, which is then immersed, up to the neck, in a pre-heated oil bath. The oil bath is maintained at 140° C. using a temperature controlled hotplate. After the MePEG and DL-lactide have melted and reached 140° C., 2 mL of 95% stannous octoate (catalyst) is added to the flask. The flask is vigorously shaken immediately after the addition to ensure rapid mixing and is then returned to the oil bath. The reaction is allowed to proceed for 6 hours with heat and stirring. The liquid polymer is then poured into a stainless steel tray, covered and left in the fume hood overnight (about 16 hours). The polymer solidifies in the tray. The top of the tray is sealed using parafilm. The sealed tray containing the polymer is placed in a freezer at −20° C.±5° C. for 0.5 hour. The polymer is then removed from the freezer and transferred to glass storage bottles and stored at 2-8° C.

Preparation of Micellar Batimistat (Batimistat/Polymer Matrix)

Reaction glassware is washed and rinsed with Sterile Water for Irrigation USP, dried at 37° C., followed by depyrogenation at 250° C. for at least 1 hour. First, a phosphate buffer, 0.08M, pH 7.6 is prepared. The buffer is dispensed at the volume of 1 mL per vial. The vials are heated for 2 hours at 90° C. to dry the buffer. The temperature is then raised to 160° C. and the vials are dried for an additional 3 hours.

The polymer is dissolved in THF at 10% w/v concentration with stirring and heat. The polymer solution is then centrifuged at 3000 rpm for 30 minutes. The supernatant is poured off and set aside. Additional THF is added to the precipitate and centrifuged a second time at 3000 rpm for 30 minutes. The second supernatant is pooled with the first supernatant. BATIMASTAT is weighed and then added to the supernatant pool. The solution is brought to the final desired volume with THF to make a 9.9% polymer solution containing 1.1% BATIMASTAT.

To manufacture development batches of final product vials, the micellar Batimistat is dispensed into the vials containing dried phosphate buffer at a volume of 1 mL per vial. The vials are placed in a vacuum oven at 50° C. The vacuum is set at −80 kPa and the vials remain in the oven overnight (15 to 24 hours). The vials are capped with Teflon faced gray butyl stoppers and sealed with aluminum seals. The BATIMASTAT/polymer matrix is sterilized using 2.5 Mrad γ-ray irradiation. Each vial contains approximately 11 mg BATIMASTAT, 99 mg polymer, and 11 mg phosphate salts. The vials are stored at 2° to 8° C. until constitution.

Preparation of the Micellar Batimistat/Collagen Gel

In a sterile biological safety cabinet, two milliliters sterile saline is added to a vial that contained approximately 11 mg BATIMASTAT, 99 mg polymer, and 11 mg phosphate salts (as prepared above). The contents of the vial are dissolved in 2 mL sterile saline by placing the vial in a water bath at 37 ° C. for approx. 30 minutes with periodic vortexing. Using a sterile 1 mL syringe, a 1 mL aliquot of the micellar BATIMASTAT solution is withdrawn from the vial and was injected into 29 mL collagen gel. The sample is mixed to produce a homogeneous solution of the micellar BATIMASTAT in the collagen gel. The sample is then loaded into 1 mL syringes for use in the in vivo experiments.

### Example 10

### Preparation of a 2 Component Micellar Kit

Preparation of Freeze Dried Micellar BATIMASTAT

A solid composition capable of forming micelles upon constitution with an aqueous collagen-containing medium is prepared as follows:

41.29 g of MePEG (MW =2,000 g/mol) is combined with 412.84 g of 60:40 MePEG:poly(DL-lactide) diblock copolymer (see the example provided above) in a stainless steel beaker, heated to 75° C. in a mineral oil bath and stirred by an overhead stirring blade. Once a clear liquid is obtained, the mixture is cooled to 55° C. To the mixture is added a 200 ml solution of 45.87 g BATIMASTAT in tetrahydrofuran. The solvent is added at approximately 40 ml/min and the mixture stirred for 4 hours at 55° C. After mixing for this time, the liquid composition is transferred to a stainless steel pan and placed in a forced air oven at 50° C. for about 48 hours to remove residual solvent. The composition is then cooled to ambient temperature and is allowed to solidify to form Batimistat-polymer matrix.

A phosphate buffer is prepared by combining 237.8 g of dibasic sodium phosphate heptahydrate, 15.18 g of monobasic sodium phosphate monohydrate in 1600 ml of water. To the phosphate buffer, 327 g of the BATIMASTAT-polymer matrix is added and stirred for 2 hours to dissolve the solids. After a clear solution is achieved, the volume is adjusted to 2000 ml with additional water. Vials are filled with 15 ml aliquots of this solution and freeze dried by cooling to −34° C., holding for 5 hours, heating to −16° C. while reducing pressure to less than 0.2 mm Hg, holding for 68 hours, heating to 30° C. while maintaining low pressure, followed by holding for a further 20 hours. The result is a freeze-dried matrix that could be reconstituted to form a clear micellar solution.

Preparation of 2 Component Kit

40 mg of the freeze-dried micellar BATIMASTAT material is weighed into a capped 1 mL syringe. The plunger is replaced and the syringe is sealed in a plastic pouch using a heat sealer. The sample is sterilized using 2.5 Mrad γ-ray irradiation. Just prior to application, the plastic pouch containing the sterilized freeze-dried material is opened and connected to a dual syringe connector. A syringe containing 2 mL 3.5% bovine collagen (95% type I and 5% Type III) is attached to the remaining end of the dual syringe connector. The plunger of the syringe containing the collagen material is pushed in order to transfer the collagen material into the syringe containing the micellar material. The material is passed rapidly from one syringe to the other until a homogeneous solution is obtained. The material is then transferred into the syringe that originally contained the collagen. This syringe is disconnected from the connector and a 30-gauge needle is connected to the syringe. The material is now ready for application.

### Example 11

### Preparation of a 2 Component Microsphere Kit

40 mg of the freeze-dried microsphere BATIMASTAT material is weighed into a capped 1 mL syringe. The plunger is replaced and the syringe is sealed in a plastic pouch using a heat sealer. The sample is sterilized using 2.5 Mrad γ-ray irradiation. Just prior to application, the plastic pouch containing the sterilized freeze-dried material is opened and connected to a dual syringe connector. A syringe containing 2 mL 3.5% bovine collagen (95% type I and 5% Type III) is attached to the remaining end of the dual syringe connector. The plunger of the syringe containing the collagen material is pushed in order to transfer the collagen material into the syringe containing the micellar material. The material is passed from one syringe to the other until a homogeneous solution is obtained. The material is then transferred into the syringe that originally contained the collagen. This syringe is disconnected from the connector and a 30-gauge needle is connected to the syringe. The material is now ready for application.

### Example 12

### Liposomal Preparations

MLV Liposomes

A total of 100 mg of egg phosphatidylcholine (Avanti Polar Lipids, Alabaster, Ala.) and cholesterol (Sigma Chemical Co., St. Louis, Mo.) [5:1 molar ratio] are added to 5 mL dichloromethane in a 50 mL round bottom flask. Once dissolved, 3 mg BATIMASTAT is added to the solution. The solvent is removed under slight vacuum using the rotary evaporator. The lipid-drug mixture is dried overnight under vacuum. 5 mL 0.9% NaCl solution is added to the dried lipid-drug mixture. The solution is gently rotated for 1 hour using a rotary evaporator and a water bath setting of 37° C. When 5% maltose is added to the 0.9% NaCl constitution solution, the samples are frozen in acetone dry ice and are freeze-dried to produce a solid product.

Depending on the specific dose required, a certain amount of the freeze-dried microsphere BATIMISTAT material (prepared as described above) is weighed into a capped 1 mL syringe. The plunger is replaced and the syringe is sealed in a plastic pouch using a heat sealer. The sample is sterilized using 2.5 Mrad γ-ray irradiation. Just prior to application, the plastic pouch containing the sterilized freeze-dried material is opened and connected to a dual syringe connector. A syringe containing 3.5% bovine collagen (95% type I and 5% Type III) is attached to the remaining end of the dual syringe connector. The plunger of the syringe containing the collagen material is pushed in order to transfer the collagen material into the syringe containing the micellar material. The material is passed from one syringe to the other until a homogeneous solution is obtained. The material is then transferred into the syringe that originally contained the collagen. This syringe is disconnected from the connector and a 30-gauge needle is connected to the syringe. The material is now ready for application.

SUV Liposomes

The liposomes prepared above are size reduced by placing the sample in an ultrasonic bath (45° C.) for 10 minutes. The solution changed from a opaque—milky solution to a transparent solution with a blue tinge. This solution is either used as is or is freeze-dried to produce a solid product. The solid product can be used to prepare a collagen solution in a similar manner to that described above.

SUV Liposomes

The liposomes prepared above are size reduced by placing the sample in an ultrasonic bath (45° C.) for 10 minutes. The solution changed from a opaque—milky solution to a transparent solution with a blue tinge. This solution is either used as is or is freeze-dried to produce a solid product. The solid product can be used to prepare a collagen solution in a similar manner to that described above.

### Example 13

### Hydroxyproline Assay for Assessment of Collagen Degradation

Collagen is the only protein containing 3- and 4-hydroxyproline and thus the effect of a drug formulation on collagen degradation can be quantified by the measurement of hydroxyproline following treatment with a drug-loaded formulation at various time points. Human dermal fibroblasts are grown in culture media for 3 weeks in the presence of vitamin C to form a three dimensional collagen matrix. Various concentrations of drug formulations can be aliquoted onto the collagen matrix along with various concentrations of collagen degrading enzymes. The duration of drug incubation can be altered. Cell supernatants are collected in 0.1 M NaCl, 5 mM NaHCO₃ and hydrolyzed in 6N HCL for 16 hours at 110° C. Samples are then vacuum dried and reconstituted in stock buffer diluted ten-fold with H₂O. Stock buffer consists of a 1 L solution containing 50 g of citric acid, 12 mL of glacial acetic acid, 120 g of sodium acetate and 34 g of NaOH.

Each sample is tested in triplicate by aliquoting 100 μL of the sample in a 96-well plate with 50 μL of Chloramine-T reagent (1.41 g of Chloramine-T dissolved 20.7 mL of H₂O, 26 mL of n-propanol, and stock buffer) and 50 μL of dimethylaminobenzaldehyde reagent (15 g of p-dimethylaminobenzaldehyde in 60 mL of n-propanol and 26 mL of 60% perchloric acid added slowly). The plate is incubated at 60° C. for 15 minutes and placed at 8-10° C. for 5 minutes. Optical density is read immediately on microplate spectrophotometer at 550 nm absorbance. Absorbance over triplicate wells is averaged after subtracting background and concentration values are obtained from the hydroxyproline standard curve (0-5 μg). The amount of hydroxyproline measured is a determination of collagen degradation. A reduction in the amount of hydroxyproline following incubation with a drug formulation indicates a reduction in the degradation of collagen.

This application claims the benefit of U.S. Provisional Patent Application No. 60/436,806 filed Dec. 27, 2002, wherein this provisional application is incorporated herein by reference in its entirety. All of the U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

1. A composition comprising collagen, an MMPI, and hydroxyapatite.

2. The composition of claim 1 wherein the MMPI is a Tissue Inhibitor of Matrix Metalloproteinase (TIMP).

3. The composition of claim 2 wherein the TIMP is TIMP-1 or TIMP-2.

4. The composition of claim 2 wherein the TIMP is TIMP-3 or TIMP-4.

5. The composition of claim 1 wherein the MMPI is tetracycline, or an analog or derivative thereof.

6. The composition of claim 5 wherein the MMPI is tetracycline.

7. The composition of claim 6 wherein the tetracycline is minocycline or doxycline.

8. The composition of claim 1 wherein the MMPI is a hydroxamate.

9. The composition of claim 8 wherein the hydroxamate is BATIMASTAT, MARIMASTAT, or TROCADE.

10. The composition of claim 1 wherein the MMPI is RO-1130830, CGS-27023A or BMS-275291.

11. The composition of claim 1 wherein the MMPI is a polypeptide inhibitor.

12. The composition of claim 11 wherein the polypeptide inhibitor is an inhibitor of a metalloprotease maturase.

13. The composition of claim 1 wherein the MMPI is a mercapto-based compound.

14. The composition of claim 1 wherein the MMPI is a bisphosphonate with structure (I): 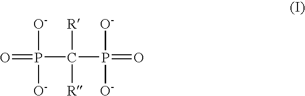
wherein R′ and R″ are independently a hydrogen, a halogen, a hydroxy, an amino group or a substituted derivative thereof, a thio group or a substituted derivative thereof, or an alkyl, alkanyl, alkenyl, alkynyl, alkyldiyl, alkyleno, heteroalkyl, heteroalkanyl, heteroalkenyl, heteroalkanyl, heteroalkyldiyl, heteroalkyleno, aryl, arylalkyl, heteroaryl, or heteroarylalkyl group, or a substituted derivative thereof.

15. The composition of claim 14 wherein the MMPI is a bisphosphonate, and wherein R′ and R″ is hydroxy, hydrogen, or chlorine.

16. The composition of claim 1 comprising at least two MMPIs.

17. The composition of claim 16 wherein the at least two MMPIs comprise a tetracycline, or an analog or derivative thereof and a bisphosphonate.

18. The composition of claim 16 wherein the at least two MMPIs comprise a tetracycline, or an analog or derivative thereof and a hydroxymate.

19. A composition comprising collagen, at least one metalloprotease inhibitor (MMPI), and at least one polymer.

20. The composition of claim 19 wherein the polymer is biodegradable.

21. The composition of claim 20 wherein the polymer is a biodegradable polymer selected from the group consisting of albumin, gelatin, starch, cellulose, dextrans, polysaccharides, fibrinogen, poly (esters), poly (D,L lactide), poly (D,L-lactide-co-glycolide), poly (glycolide), poly(ε-caprolactone), poly (hydroxybutyrate), poly (alkylcarbonate), poly(anhydrides), and poly (orthoesters), and copolymers and blends thereof.

22. The composition of claim 19 wherein the polymer is a non-biodegradable polymer selected from the group consisting of an ethylene oxide and propylene oxide copolymer, an ethylene vinyl acetate copolymer, silicone rubber, a poly (methacrylate) based polymer, and a poly (acrylate) based polymer.

23. The composition of claim 1 wherein the collagen is type I or type II collagen.

24. The composition of claim 1 wherein the collagen is type III or type IV collagen.

25. The composition of claim 1, wherein the composition is sterile.

26. The composition of claim 1, further comprising a bone morphogenic protein.

27. The composition of claim 26 wherein the bone morphogenic protein is BMP-2 or BMP-8.

28. A method for augmenting bone or replacing lost bone, comprising, delivering to a patient at a desired location a composition claim 1.

29. A medical device, comprising a collagen sponge and an MMPI.

30. The medical device of claim 29 wherein the MMPI is a Tissue Inhibitor of Matrix Metalloproteinase (TIMP).

31. The medical device of claim 30 wherein the TIMP is TIMP-1 or TIMP-2.

32. The medical device of claim 30 wherein the TIMP is TIMP-3 or TIMP-4.

33. The medical device of claim 29 wherein the MMPI is tetracycline, or an analog or derivative thereof.

34. The medical device of claim 33 wherein the MMPI is tetracycline.

35. The medical device of claim 34 wherein the tetracycline is minocycline or doxycline.

36. The medical device of claim 29 wherein the MMPI is a hydroxamate.

37. The medical device of claim 36 wherein the hydroxamate is BATIMASTAT, MARIMASTAT, or TROCADE.

38. The medical device of claim 29 wherein the MMPI is RO-1 130830, CGS-27023A , or BMS-275291.

39. The medical device of claim 29 wherein the MMPI is a polypeptide inhibitor.

40. The medical device of claim 39 wherein the polypeptide inhibitor is an inhibitor of a metalloprotease maturase.

41. The medical device of claim 29 wherein the MMPI is a mercapto-based compound.

42. The medical device of claim 29 wherein the MMPI is a bisphosphonate with structure (I): 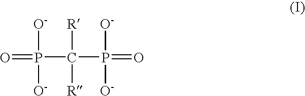
wherein R′ and R″ are independently a hydrogen, a halogen, a hydroxy, an amino group or a substituted derivative thereof, a thio group or a substituted derivative thereof, or an alkyl, alkanyl, alkenyl, alkynyl, alkyldiyl, alkyleno, heteroalkyl, heteroalkanyl, heteroalkenyl, heteroalkanyl, heteroalkyldiyl, heteroalkyleno, aryl, arylalkyl, heteroaryl, or heteroarylalkyl group, or a substituted derivative thereof.

43. The medical device of claim 42 wherein the MMPI is a bisphosphonate, and wherein R′ and R″ is hydroxy, hydrogen, or chlorine.

44. The medical device of claim 29 comprising at least two

45. The medical device of claim 29, further comprising at least one polymer.

46. The medical device of claim 45 wherein the polymer is biodegradable.

47. The medical device of claim 46 wherein the biodegradable polymer is selected from the group consisting of albumin, gelatin, starch, cellulose, dextrans, polysaccharides, fibrinogen, poly (esters), poly (D,L lactide), poly (D,L-lactide-co-glycolide), poly (glycolide), poly(ε-caprolactone), poly (hydroxybutyrate), poly (alkylcarbonate), poly(anhydrides), and poly (orthoesters), and copolymers and blends thereof.

48. The medical device of claim 45 wherein the polymer is non-biodegradable.

49. The medical device of claim 48 wherein the non-biodegradable polymer is selected from the group consisting of an ethylene oxide and propylene oxide copolymer, an ethylene vinyl acetate copolymer, silicone rubber, a poly (methacrylate) based polymer, and a poly (acrylate) based polymer.

50. The medical device of claim 29 wherein the collagen is type I or type II collagen.

51. The medical device of claim 29 wherein the collagen is type III or type IV collagen.

52. The medical device of claim 29 wherein the medical device is sterile.

53. The medical device of claim 29, further comprising a bone morphogenic protein.

54. The medical device of claim 53 wherein the bone morphogenic protein is BMP-2 or BMP-8.

55. The medical device of claim 29, further comprising hydroxyapatite.

56. A method for surgically fusing a portion of a spine comprising:
removing a portion of a degenerated disc from the spine of a patient to form a disc space; and
inserting into the disc space the medical device of claim 29.

57. A method for surgically fusing a portion of a spine comprising:
removing a portion of a degenerated disc from the spine of a patient to form a disc space; and
inserting into the disc space the medical device of claim 55.

58. The method of claim 54 wherein the MMPI is tetracycline.

59. The method of claim 54 wherein the MMPI is a chemically modified tetracycline.

60. The method of claim 54 wherein the MMPI is BATIMISTAT or MARIMISTAT.

61. The method of claim 59 wherein the device comprises 0.001 % to 15% of the MMPI by weight.

62. A method of treating periodontal disease comprising:
placing a dental implant comprising collagen and an MMPI between gingival tissue and a debrided periodontal defect in the mouth of a patient.

63. A method of treating periodontal disease comprising:
placing a dental implant comprising collagen, an MMPI, and hydroxyapatite between gingival tissue and a debrided periodontal defect in the mouth of a patient.

64. A method of treating gastroesophageal reflux disease comprising injecting a composition into the vicinity of the lower esophageal sphincter of a patient, wherein the composition comprises collagen and an MMPI.

65. A method of treating fecal incontinence comprising injecting a composition into the vicinity of the anal sphincter of a patient, wherein the composition comprises collagen and an MMPI.

66. A medical device comprising the composition of claim 1, wherein the device is selected from the group consisting of surgical meshs, surgical slings, surgical patches, dental implants, skin grafts, corneal shields, and glaucoma drainage devices.

67. A method of reinforcing soft tissue during an operative repair comprising attaching to the soft tissue a surgical patch, wherein the patch comprises collagen and an MMPI.

68. The method of claim 65 wherein the operative repair is an abdominal or thoracic wall repair, a hernia repair, a suture line reinforcement, an ostomy reinforcement, or a tissue flap donor site repair.

69. The method of claim 67 wherein the operative repair is a repair of a tendon, ligament, or cartilage.

70. A method of improving drainage of the aqueous humor following a sclerectomy comprising inserting into a subscleral drainage channel a glaucoma drainage device, wherein the device comprises collagen and an MMPI.

71. A method of improving wound healing comprising applying a wound dressing to a wound surface, wherein wound dressing comprises collagen and an MMPI.

72. A method of improving post-operative healing of cornea following cataract surgery, comprising applying a corneal shield to scleral or conjunctival tissue, wherein corneal shield comprises collagen and an MMPI.

73. The method of claim 62 wherein the MMPI is a Tissue Inhibitor of Matrix Metalloproteinase (TIMP).

74. The method of claim 62 wherein the MMPI is TIMP-1 or TIMP-2.

75. The method of claim 62 wherein the MMPI is TIMP-3 or TIMP-4.

76. The method of claim 62 wherein the MMPI is tetracycline, or an analog or derivative thereof.

77. The method of claim 62 wherein the MMPI is tetracycline.

78. The method of claim 62 wherein the MMPI is minocycline or doxycline.

79. The method of claim 62 wherein the MMPI is a hydroxamate.

80. The method of claim 62 wherein the MMPI is BATIMASTAT, MARIMASTAT, or TROCADE.

81. The method of claim 62 wherein the MMPI is RO-1 130830, CGS-27023A or BMS-275291.

82. The method of claim 62 wherein the MMPI is a polypeptide inhibitor.

83. The method of claim 62 wherein the MMPI is an inhibitor of a metalloprotease maturase.

84. The method of claim 62 wherein the MMPI is a mercapto-based compound.

85. The method of claim 62 wherein the MMPI is a bisphosphonate with structure (I): 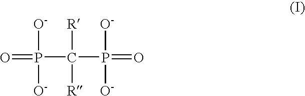
wherein R′ and R″ are independently a hydrogen, a halogen, a hydroxy, an amino group or a substituted derivative thereof, a thio group or a substituted derivative thereof, or an alkyl, alkanyl, alkenyl, alkynyl, alkyldiyl, alkyleno, heteroalkyl, heteroalkanyl, heteroalkenyl, heteroalkanyl, heteroalkyldiyl, heteroalkyleno, aryl, arylalkyl, heteroaryl, or heteroarylalkyl group, or a substituted derivative thereof.

86. The method of claim 62 wherein the MMPI a bisphosphonate with structure (I): 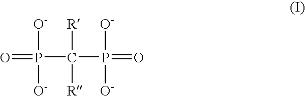
and R′ and R″ are independently hydroxy, hydrogen, or chlorine.

87. The method of claim 62 wherein at least MMPIs are utilized in the method.

88. The method of claim 62 wherein the MMPI comprises both a tetracycline or an analog or derivative thereof, and a bisphosphonate.

89. The method of claim 62 wherein the MMPI comprises both a tetracycline or an analog or derivative thereof, and a hydroxymate.

90. An implant comprising an orthopedic implant comprising collagen and an MMPI.

91. The implant of claim 90 in the form of bone graft matrix.

92. The implant of claim 90 where the implant is a spinal fusion device.

93. An implant comprising a surgical mesh that comprises collagen, and an MMPI.

94. An implant comprising a sling that comprises collagen, and an MMPI.

95. An implant comprising a patch that comprises collagen, and an MMPI.

96. An implant comprising a dental implant comprising collagen, and an MMPI.

97. An implant comprising an artificial skin graft comprising collagen, and an MMPI.

98. An implant comprising a corneal shield comprising collagen, and an MMPI.

99. An implant comprising a glaucoma drainage device comprising collagen, and an MMPI.

100. An implant comprising a bulking agent comprising collagen, and an MMPI.

101. The implant of claim 100 formulated for management of GERD.

102. The implant of claim 100 formulated for management of fecal incontinence.

103. The implant of claim 90 wherein the MMPI is a Tissue Inhibitor of Matrix Metalloproteinase (TIMP).

104. The implant of claim 90 wherein the MMPI is TIMP-1 or TIMP-2.

105. The implant of claim 90 wherein the MMPI is TIMP-3 or TIMP-4.

106. The implant of claim 90 wherein the MMPI is tetracycline, or an analog or derivative thereof.

107. The implant of claim 90 wherein the MMPI is tetracycline.

108. The implant of claim 90 wherein the MMPI is minocycline or doxycline.

109. The implant of claim 90 wherein the MMPI is a hydroxamate.

110. The implant of claim 90 wherein the MMPI is BATIMASTAT, MARIMASTAT, or TROCADE.

111. The implant of claim 90 wherein the MMPI is RO-1 130830, CGS-27023A, or BMS-275291.

112. The implant of claim 90 wherein the MMPI is a polypeptide inhibitor.

113. The implant of claim 90 wherein the MMPI is an inhibitor of a metalloprotease maturase.

114. The implant of claim 90 wherein the MMPI is a mercapto-based compound.

115. The implant of claim 90 wherein the MMPI is a bisphosphonate with structure (I): 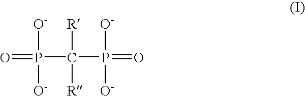
wherein R′ and R″ are independently a hydrogen, a halogen, a hydroxy, an amino group or a substituted derivative thereof, a thio group or a substituted derivative thereof, or an alkyl, alkanyl, alkenyl, alkynyl, alkyldiyl, alkyleno, heteroalkyl, heteroalkanyl, heteroalkenyl, heteroalkanyl, heteroalkyldiyl, heteroalkyleno, aryl, arylalkyl, heteroaryl, or heteroarylalkyl group, or a substituted derivative thereof.

116. The implant of claim 90 wherein the MMPI a bisphosphonate with structure (I): 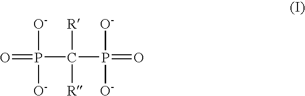
sand R′ and R″ are independently hydroxy, hydrogen, or chlorine.

117. The implant of claim 90 comprising at least MMPIs.

118. The implant of claim 90 comprising both a tetracycline or an analog or derivative thereof, and a bisphosphonate.

119. The implant of claim 90 comprising both a tetracycline or an analog or derivative thereof, and a hydroxymate.
